SEQUENCE LISTING (1 sequence)

SEQ ID NO: 1 - DNA, 2977 bp, Homo Sapiens
gacaagcttgcatgcctgcaggtcgactctagaggatccgccgccaccatggcgccccgcagcgcccggcgacccctgctgctgctactgctgttgctgctgctcggcctcatgcattgtgcgtcagcagcaatgtttatggtgaaaaatggcaacgggaccgcgtgcataatggccaacttctctgctgccttctcagtgaactacgacaccaagagtggccctaagaacatgacccttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgtccagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagccggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagcctcgagatggagtcgcgcggtcgccgttgtcccgaaatgatatccgtactgggtcccatttcggggcacgtgctgaaagccgtgtttagtcgcggcgacacgccggtgctgccgcacgagacgcgactcctgcagacgggtatccacgtgcgcgtgagccagccctcgctgatcctggtgtcgcagtacacgcccgactcgacgccatgccaccgcggcgacaatcagctgcaggtgcagcacacgtactttacgggcagcgaggtggagaacgtgtcggtcaacgtgcacaaccccacgggccggagcatctgccccagccaagagcccatgtcgatctatgtgtacgcgctgccgctcaagatgctgaacatccccagcatcaacgtgcaccactacccgtcggcggccgagcgcaaacaccgacacctgcccgtagctgacgctgtgattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctgacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctattgatgaacgggcagcagatcttcctggaggtgcaagcgatacgcgagaccgtggaactgcgtcagtacgatcccgtggctgcgctcttctttttcgatatcgacttgctgctgcagcgcgggcctcagtacagcgaacaccccaccttcaccagccagtatcgcatccagggcaagcttgagtaccgacacacctgggaccggcacgacgagggtgccgcccagggcgacgacgacgtctggaccagcggatcggactccgacgaggaactcgtaaccaccgagcgcaagacgccccgcgttaccggcggcggcgccatggcgggcgcctccacttccgcgggccgcaaacgcaaatcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagacgccttgcccggggaattcacgctgatccccatcgctgtgggtggtgccctggcggggctggtcctcatcgtcctcatcgcctacctcgtcggcaggaagaggagtcacgcaggctaccagactatctagtaagatctaattcttaatt

(19) United States
(12) Patent Application Publication
(10) Pub. No.: US 2024/0299516 A1
(43) Pub. Date: Sep. 12, 2024

(54) MULTILAMELLAR RNA NANOPARTICLE VACCINE AGAINST CANCER

(71) Applicant: UNIVERSITY OF FLORIDA RESEARCH FOUNDATON, INC., Gainesville, FL (US)

(72) Inventors: Elias Sayour, Newberry, FL (US); Hector Ruben Mendez-Gomez, Gainesville, FL (US); Duane A. Mitchell, Gainesville, FL (US)

(21) Appl. No.: 18/268,725

(22) PCT Filed: Dec. 20, 2021
(86) PCT No.: PCT/US21/64398

§ 371 Date: Jun. 21, 2023

Related U.S. Application Data
(60) Provisional application No. 63/128,611, filed on Dec. 21, 2020.

(51) Int. Cl. A61K 39/00; A61K 9/127; A61P 35/00

(52) U.S. Cl. CPC A61K 39/0011; A61K 9/1272; A61P 35/00; A61K 2039/53; A61K 2039/545; A61K 2039/585

(57) ABSTRACT

The disclosure provides a method of treating cancer in a human subject. The method comprises, e.g., administering a dose of nanoparticles every two weeks for an initial treatment period, then administering a dose of nanoparticles once a month for a subsequent treatment period. The nanoparticles comprise a positively-charged surface and an interior comprising (i) a core and (ii) at least two nucleic acid layers, each nucleic acid layer being positioned between a cationic lipid bilayer, wherein the nucleic acids are derived from a cancer cell. The dose comprises about 0.00050 mg/kg to about 1.5 mg/kg of nucleic acid.

### CROSS-REFERENCE TO RELATED APPLICATION AND INCORPORATION BY REFERENCE OF MATERIAL SUBMITTED ELECTRONICALLY

This application claims the benefit of priority to U.S. Provisional Patent Application No. 63/128,611, filed Dec. 21, 2020, which is hereby incorporated by reference in its entirety.

### GOVERNMENT SUPPORT CLAUSE

This invention was made with government support under grant number K08 CA 199224 awarded by the National Institutes of Health, and grant number W81XWH-17-1-0510 awarded by the U.S. Army Medical Research Acquisition Activity. The government has certain rights in the invention.

### FIELD

The disclosure relates to a method of treating cancer using nanoparticles comprising nucleic acids derived from a cancer.

Incorporated by reference in its entirety is a computer-readable nucleotide/amino acid sequence listing submitted concurrently herewith and identified as follows: 4,262 byte ASCII (Text) file named “55682_Seqlisting.txt”; created on Dec. 20, 2021.

### BACKGROUND

Due to severe and non-specific deleterious effects of radiation and chemotherapy, targeted therapies capable of selectively killing tumor cells in patients with cancers, including glioblastoma (GBM), are essential (see, e.g., Stupp et al., The New England Journal of Medicine. 2005; 352(10):987-96). Tumor-specific immunotherapy can be harnessed to eradicate malignant brain tumors with exquisite precision and without collateral damage to normal tissue. Immunotherapy relies on the cytotoxic potential of activated T cells, which scavenge to recognize and reject tumor associated or specific antigens (TAAs or TSAs). Unlike most drug agents, activated T cells can traverse the blood brain barrier (BBB) via integrin (i.e., LFA-1, VLA-4) binding of ICAMs/VCAMs (Sampson et al., Neuro Oncol. 2011; 13(3):324-33; Ransohoff et al., Nature Reviews Immunology. 2003; 3(7):569-81; Miao et al., PloS one. 2014; 9(4):e94281). T cells can be ex vivo activated in co-culture with dendritic cells (DCs) presenting TAAs/TSAs (Mitchell et al., Nature. 2015; 519(7543):366-9) or through transduction with a chimeric antigen receptor (CAR) (Grupp et al., The New England journal of medicine. 2013; 368(16): 1509-18). Alternatively, T cells can be endogenously activated using cancer vaccines; but, in a randomized phase III trial for patients with primary GBM, peptide vaccines targeting the tumor specific EGFRVIII surface antigen failed to mediate enhanced survival benefits over control vaccines (Weller et al., The Lancet Oncology. 2017; 18(10): 1373-85). The EGFRVIII vaccine's failure to mediate anti-tumor efficacy highlights the challenge of therapeutic cancer vaccines. While prophylactic cancer vaccines work to prevent malignancies (i.e., HPV vaccine to prevent cervical cancer), the vaccines require several boosts over months to years to confer protection in immune-replete patients. Furthermore, therapeutic cancer vaccines must induce immunologic response much more rapidly against malignancies (i.e., GBM) that are rapidly evolving (Sayour et al., Int J Mol Sci. 2018; 19(10)). Moreover, GBMs are a highly invasive and heterogenous tumors associated with profound systemic/intratumoral suppression that can stymie a nascent immunotherapeutic response (Chongsathidkiet et al., Nature Medicine. 2018; 24(9): 1459-68; Learn et al., Clinical cancer research: an official journal of the American Association for Cancer Research. 2006; 12(24):7306-15).

RNA vaccines have been studied as an alternative to cell-based therapeutics. See, e.g., Reichmuth et al., Ther Deliv 7(5): 319-334 (2016). RNA vaccines have several advantages over traditional modalities. RNA has potent effects on both the innate and adaptive immune system. RNA can act as a toll-like receptor (TLR) agonist for receptors 3, 7, and 8, inducing potent TLR dependent innate immunity. RNA can also stimulate intracellular pathogen recognition receptors (i.e., melanoma differentiation antigen 5 (MDA-5) and retinoic acid inducible gene I (RIG-I)) and culminates in activating both helper-CD4 and cytotoxic CD8 T cell responses. Unlike DNA vaccines mired by having to cross both cellular and nuclear membranes, RNA only requires access to the cytoplasm and carries a significant safety advantage since it cannot be integrated into the host-genome. RNA also bypasses MHC class restriction and can be leveraged for the population at large. However, formulations proposed for RNA vaccines have been mired by poor immunogenicity or are encumbered by the profound intratumoral and systemic immunosuppression that may stymie an activated T cell response.

### SUMMARY

Nanoparticles comprising a positively-charged surface and an interior comprising (i) a core and (ii) at least two nucleic acid layers, wherein each nucleic acid layer is positioned between a cationic lipid bilayer, mediate peripheral and intratumoral activation of dendritic cells (DCs). These multilamellar RNA NPs also demonstrate superior efficacy of multilamellar tumor specific RNA-NPs, relative to anionic LPX and RNA LPX, and demonstrate the ability to systemically activate DCs, induce antigen specific immunity, and elicit anti-tumor efficacy in vivo. The administration of multilamellar RNA NPs to diseased animals (e.g., tumor bearing mice) also led to increased survival of these animals. Without being bound to a particular theory, the multilamellar RNA nanoparticles of the present disclosure are capable of safely and effectively inducing immunity against cancers.

The disclosure provides a method of treating cancer in a human subject, the method comprising (a) administering to the human subject a dose of nanoparticles every two weeks for an initial treatment period, then (b) administering a dose of nanoparticles once a month for a subsequent treatment period. The nanoparticles comprise a positively-charged surface and an interior comprising (i) a core and (ii) at least two nucleic acid layers, each nucleic acid layer being positioned between a cationic lipid bilayer. The nucleic acids (e.g., mRNA) are derived from a cancer cell, and the dose comprises about 0.00050 mg/kg to about 1.5 mg/kg of nucleic acid.

Optionally, the nanoparticles comprise at least three nucleic acid layers or five or more nucleic acid layers, each of which is positioned between a cationic lipid bilayer. Also optionally, the nanoparticles comprise a zeta potential of about 40 mV to about 60 mV (e.g., a zeta potential of about 50 mV). The cationic lipid is DOTAP or DOTMA in various embodiments, and the nanoparticles may comprise nucleic acid molecules and cationic lipid at a ratio of about 1 to about 5 to about 1 to about 20.

In some embodiments, a single dose of nanoparticles comprises about 0.000625 mg/kg to about 0.08 mg/kg nucleic acid (e.g., about 0.000625 mg/kg of mRNA, about 0.00125 mg/kg mRNA, about 0.0025 mg/kg mRNA, about 0.005 mg/kg mRNA, about 0.01 mg/kg mRNA, about 0.02 mg/kg mRNA, about 0.04 mg/kg mRNA, or about 0.08 mg/kg mRNA). In various aspects, the nucleic acid of a dose is encapsulated into about 0.008 mg/kg to about 1.5 mg/kg of liposome material.

Optionally, the initial treatment period comprises administration of three doses over the course of about four weeks, and the subsequent treatment period comprises administration of doses over the course of about 12 months. The initial treatment period and the subsequent treatment period total about 18 months, in various embodiments.

In various aspects, the subject is suffering from a malignant brain tumor, optionally, a glioblastoma, medulloblastoma, diffuse intrinsic pontine glioma, or a peripheral tumor with metastatic infiltration into the central nervous system. The human may be an adult subject aged 21 years or older or may be a pediatric subject aged less than 21 years.

Additional embodiments and aspects of the presently disclosed pharmaceutical compositions and methods are provided below.

It should be understood that, while various embodiments in the specification are presented using “comprising” language, under various circumstances, a related embodiment may also be described using “consisting of” or “consisting essentially of” language. The disclosure contemplates embodiments described as “comprising” a feature to include embodiments which “consist of” or “consist essentially of” the feature. The term “a” or “an” refers to one or more. For example, “a nanoparticle” is understood to represent one or more nanoparticles. As such, the terms “a” (or “an”), “one or more,” and “at least one” can be used interchangeably herein. The term “or” should be understood to encompass items in the alternative or together, unless context unambiguously requires otherwise.

It should also be understood that when describing a range of values, the disclosure contemplates individual values found within the range. For example, “a pH from about pH 4 to about pH 6,” could be, but is not limited to, pH 4.2, 4.6, 5.2, 5.5, etc., and any value in between such values. In any of the ranges described herein, the endpoints of the range are included in the range. However, the description also contemplates the same ranges in which the lower and/or the higher endpoint is excluded. When the term “about” is used, it means the recited number plus or minus 5%, 10%, or more of that recited number. The actual variation intended is determinable from the context.

Additional features and variations of the invention will be apparent to those skilled in the art from the entirety of this application, including the figures and detailed description, and all such features are intended as aspects of the invention. Likewise, features of the invention described herein can be re-combined into additional embodiments that also are intended as aspects of the invention, irrespective of whether the combination of features is specified as an aspect or embodiment of the invention. Section headings are provided merely for the convenience of the reader. The entire document is intended to be related as a unified disclosure, and it should be understood that all combinations of features described herein (even if described in separate sections) are contemplated, even if the combination of features is not found together in the same sentence, or paragraph, or section of this document. Also, only such limitations which are described herein as critical to the invention should be viewed as such; variations of the invention lacking limitations which have not been described herein as critical are intended as aspects of the invention.

### BRIEF DESCRIPTION OF THE DRAWINGS

FIG. 1A is a series of illustrations of a lipid bilayer, liposome and a general scheme leading to multilamellar (ML) RNA NPs (boxed).

FIG. 1B is a pair of CEM images of uncomplexed NPs (left) and ML RNA NPs (right).

FIG. 2A is an illustration of a general scheme leading to cationic RNA lipoplexes.

FIG. 2B is an illustration of a general scheme leading to cationic RNA lipoplexes.

FIG. 2C is a graph of the % survival of mice treated with ML RNA NPs (ML RNA-NPs), RNA LPXs, anionic LPXs, or of untreated mice.

FIG. 3A is a pair of photographs of lungs of mice treated with ML RNA NPs or of untreated mice.

FIG. 3B is a graph of the % central memory T cells (CD62L+CD44+ of CD3+ cells) present in mice treated with ML RNA NPs loaded with tumor specific RNA or with ML RNA NPs with non-specific RNA (GFP RNA) or of untreated mice.

FIG. 3C is a graph of the % survival of mice treated with ML RNA NPs loaded with tumor specific RNA or with ML RNA NPs with non-specific RNA (GFP RNA) or of untreated mice.

FIG. 3D is a graph of the % survival of mice treated with ML RNA NPs loaded with tumor specific RNA or with ML RNA NPs with non-specific RNA (GFP RNA) or of untreated mice. This model is different from the one used to obtain the data of FIG. 3C.

FIGS. 4A-4D are graphs. FIG. 4A is a graph of the % expression of CD8 or CD44 and CD8 of CD3+ cells plotted as a function of time post administration of ML RNA NPs. FIG. 4B is a graph of the % expression of PDL1, MHC II, CD86 or CD80 of CD11c+ cells plotted as a function of time post administration of ML RNA NPs. FIG. 4C is a graph of the % expression of CD44 and CD8 of CD3+ cells plotted as a function of time post administration of ML RNA NPs. FIG. 4D is a graph of the % survival of a canine treated with ML RNA NPs compared to the median survival (dotted line).

FIG. 5 is a cartoon delineating the generation of personalized tumor mRNA loaded NPs. From as few as 100-500 biopsied brain tumor cells, total RNA is extracted and a cDNA library is generated from which copious amounts of mRNA (representing a personalized tumor specific transcriptome) can be amplified. Negatively charged tumor mRNA is then encapsulated into positively charged lipid NPs. NPs encapsulate RNA through electrostatic interaction and are administered intravenously (i.v.) for uptake by dendritic cells (DCs) in reticuloendothelial organs (i.e., liver spleen and lymph nodes). The RNA is then translated and processed by a DC's intracellular machinery for presentation of peptides onto MHC Class I and II molecules, which activate CD4 and CD8+ T cells.

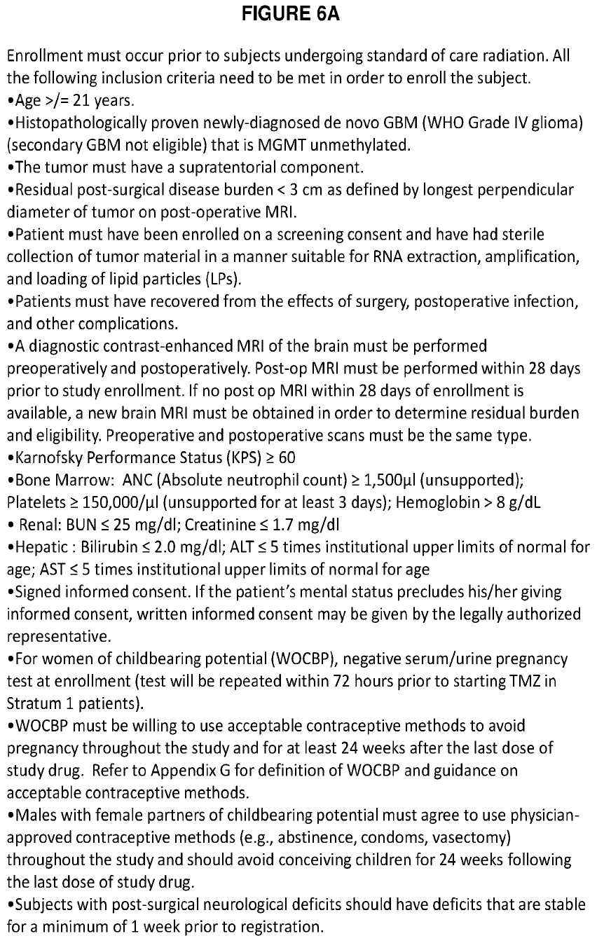

FIGS. 6A-6D are inclusion and exclusion criteria for subjects in the study referenced in the Examples.

FIG. 7 relates to an in vivo study. FIG. 7 is a graph of the percent survival of animals after the second tumor inoculation for each of the three groups of mice: two groups treated before 2^{nd} tumor inoculation with ML RNA NPs comprising non-specific RNA (RNA not specific to the tumor in the subject; Green Fluorescence Protein (GFP) or pp65) and one group treated before 2^{nd} tumor inoculation with ML RNA NPs comprising tumor specific RNA or untreated animals prior to 2^{nd} tumor inoculation. Control group survival percentage is noted as “Untreated”.

FIG. 8 is a series of images depicting the localization of anionic LPX in mice upon administration.

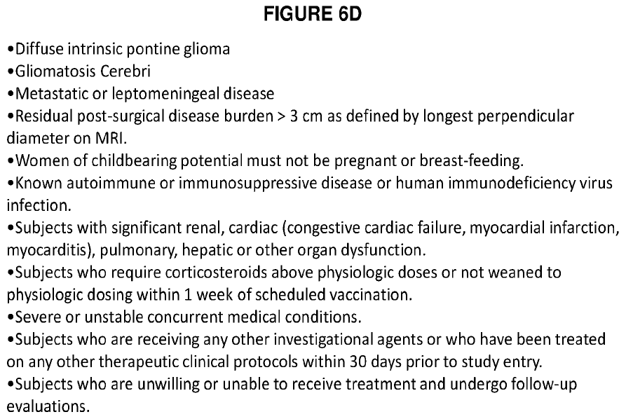

FIG. 9 demonstrates RNA-NPs improve median survival in canines with terminal gliomas. Survival of canines (boxer breed) age 9-11 years (n=5) diagnosed with terminal gliomas receiving only supportive care and weekly tumor RNA-NPs (×3) (following tumor biopsy without resection). Median survival shown as dotted line (29 days) is reported from a previous study of cerebral astrocytomas in canines receiving only supportive care. Mean survival from separate studies ranges between 60-80 days. Median one-sided exact log rank test p-value distribution with bootstrapping to account for uncertainty: *p=0.021.

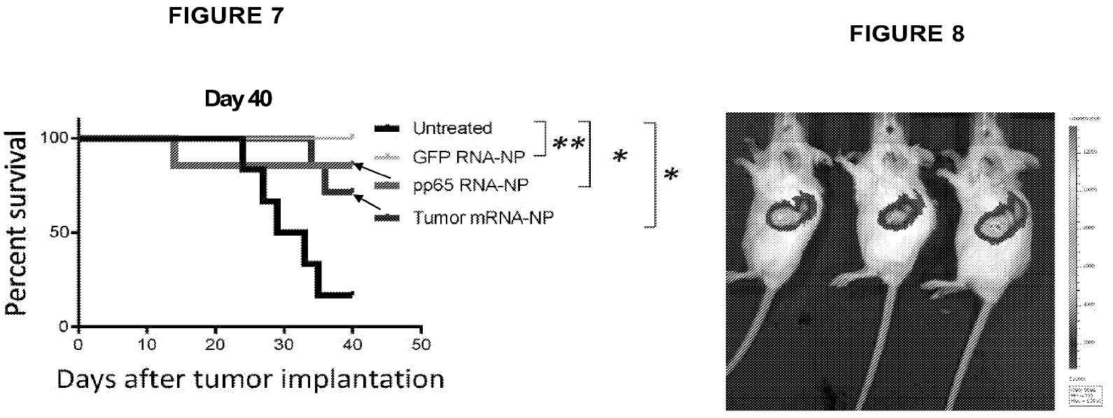

FIG. 10 demonstrates multi-lamellar RNA-NPs are superior to LPX and peptide based vaccines in eliciting antigen specific T cells. RNA/anionic lipoplex (LPX) (left) or peptide based vaccines (right) formulated in complete Freund's adjuvant (CFA) were compared with OVA specific RNA-NPs. Animals (n=5-8/group) received 10⁷ OT-Is before assessment of tetramer positive (OVA specific) T cells one week after last vaccine.

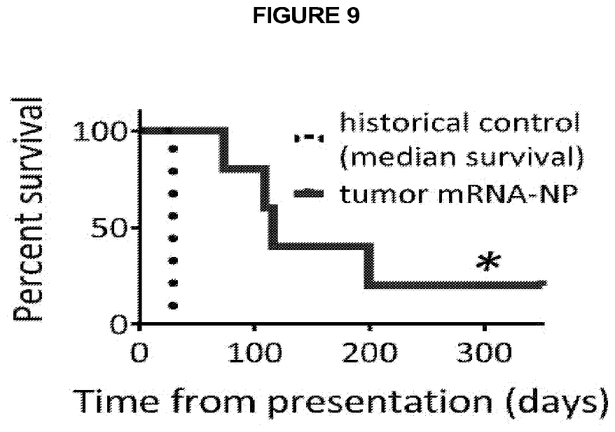

FIG. 11 demonstrates RNA-NPs induce memory re-stimulation response against CMV matrix protein pp65. Weekly pp65 RNA-NPs (×3) were administered to naïve C57/Bl/6 mice, and splenocytes were harvested one week later for culture with overlapping pp65 peptide pool and assessment of IFN-γ (*p<0.05, **p<0.01, Mann Whitney).

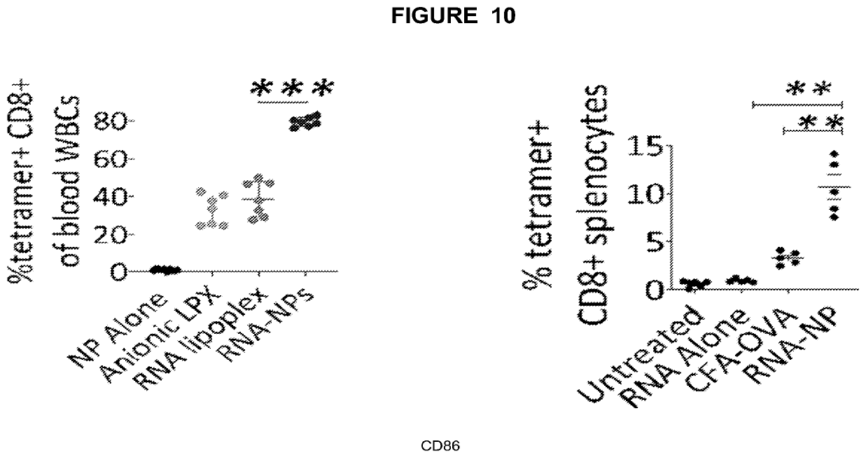

FIG. 12 demonstrates multi-lamellar tumor specific mRNA-NPs mediate superior efficacy. Different lipoplexes (LPX) or RNA-NPs were loaded with tumor specific mRNA and compared in a therapeutic lung cancer model (K7M2) (n=8/group). Each vaccine was i.v. administered weekly (×3), **p<0.01, Gehan-Wilcoxon test.

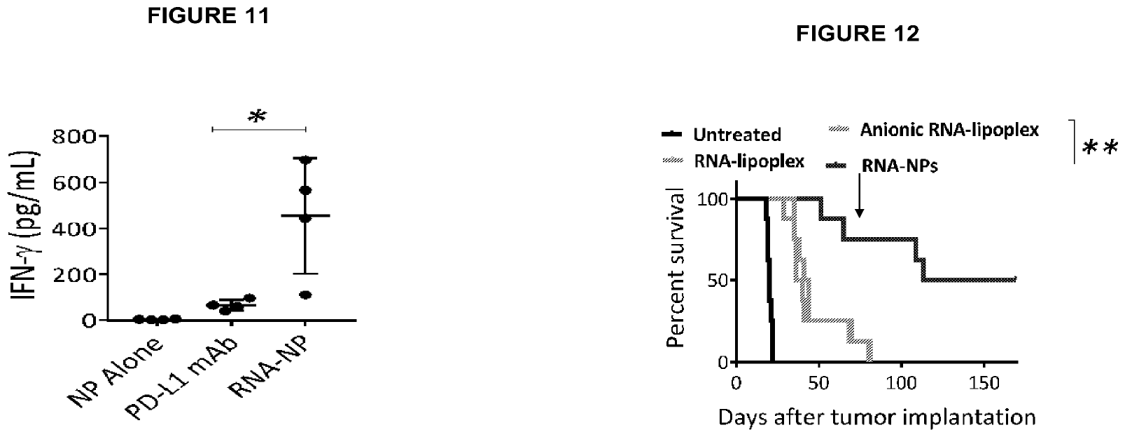

FIGS. 13A-13D demonstrate charge modified RNA-NPs can be directed to, e.g., the lung or the spleen. FIG. 13A: Cationic (˜+40 mV, left) versus anionic (˜−30 mV, right) RNA-NPs encoding for luciferase were administered to Balb/c mice by i.v. tail vein injection. Mice were injected i.p. with luciferin 6 h after RNA-NPs and imaged for bioluminescence by IVIS imaging. FIGS. 13B-13D: RNA-NPs were injected i.v. into C57Bl/6 mice (n=3-4/group). Reticuloendothelial organs (lymph nodes, spleens, and livers) were harvested within 24 h for assessment of CD11c cells expressing activation marker CD86 (*p<0.05, **p<0.01, Mann-Whitney test) from lymph nodes (FIG. 13B), splenocytes (FIG. 13C), or liver cells (FIG. 13D). The data establish that the constructs of the disclosure can be delivered to reticuloendothelial organs with only a single administration. The data also establish that constructs, if desired, may be leveraged for near immediate response in the pulmonary region.

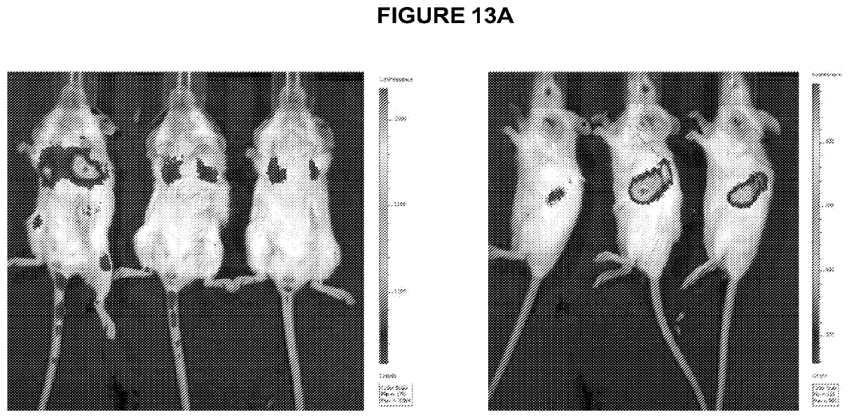

FIG. 14: RNA-NPs mediate efficacy independent of TLR7. Wild-type TLR7+/+ C57Bl/6 mice (n=8/group) were s.c. implanted with tumors derived from B16F10 and tumor volumes were compared with TLR7−/− knock out (KO) mice (n=5-8/group) on C57Bl/6 background; both groups of animals were i.v. treated with RNA-NPs weekly (×3) versus KO mice receiving NP alone (***p<0.001, two-way ANOVA).

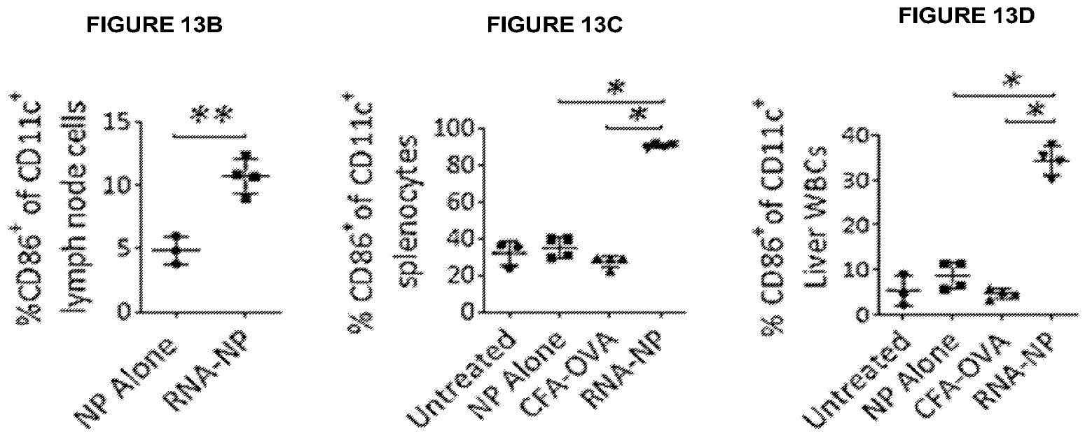

FIGS. 15A and 15B: RNA-NPs mediate IFNAR1 dependent response independent of TLR7. (FIG. 15A) K7M2 (1.25×10⁶ cells) were inoculated into the lungs of Balb/c (n=7/group) mice via tail vein injection, and i.v. treated with weekly RNA-NPs (×3) with or without biweekly IFN-α blocking antibodies (IFNAR1 mAbs), ***pWild-type TLR7+/+ C57Bl/6 mice (n=8) were implanted with B16F0 melanomas and survival outcomes were compared with TLR7−/− knock out (KO) mice (n=5/group) on C57Bl/6 background; both groups of animals were i.v. treated with RNA-NPs weekly (×3) versus KO mice receiving NP alone (*p).

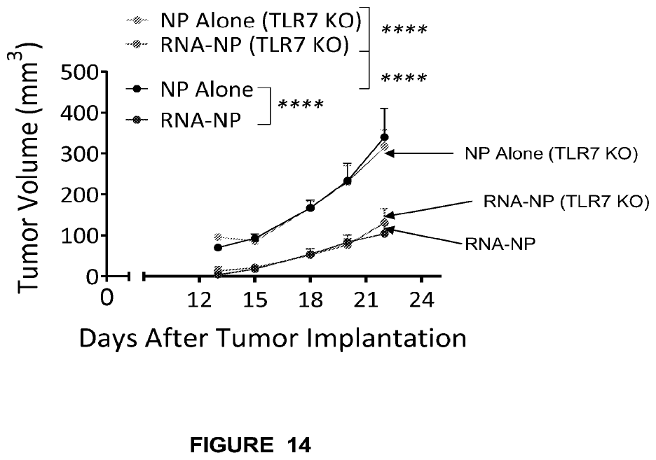

FIGS. 16A and 16B: RNA-NPs mediate memory recall response. (FIG. 16A) Balb/c mice (5-8/group) inoculated with K7M2 lung tumors were subsequently i.v. vaccinated with three weekly RNA-NPs and spleens were harvested one week after the 3rd vaccine for analysis of ex vivo memory recall response to tumor antigens (K7M2) versus control tumor (B16F0) by IFN-γ (*p<0.05, Mann Whitney test). (FIG. 16B) Long-term surviving animals previously treated with RNA-NPs (n=7), were re-challenged with i.v. administration of K7M2 tumor cells (1.25×106 cells), and compared with a new cohort of untreated mice (n=8) inoculated with K7M2 tumors (****p<0.001, Gehan-Breslow-Wilcoxon test).

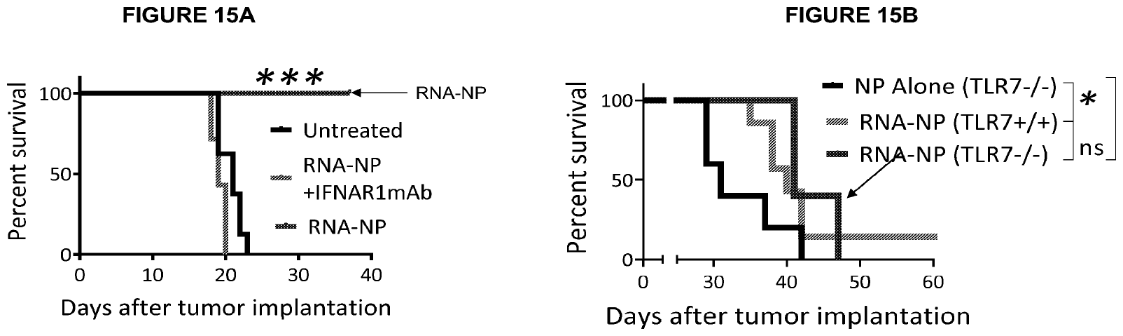

FIG. 17 illustrates that RNA-NPs with HCV's 5′ IRES and 3′polyUUU tail increase antigen specific T cells. RNA-NPs encoding OVA mRNA and comprising mRNAs containing HCV's 5′ IRES and 3′polyUUU tail were administered i.v. to naïve C57Bl/6 mice (n=3-4/group). OT-I T cells and spleens were harvested a week later for assessment of tetramer+ T cells. HCV modified RNA-NPs increase percentage of antigen specific T cells.

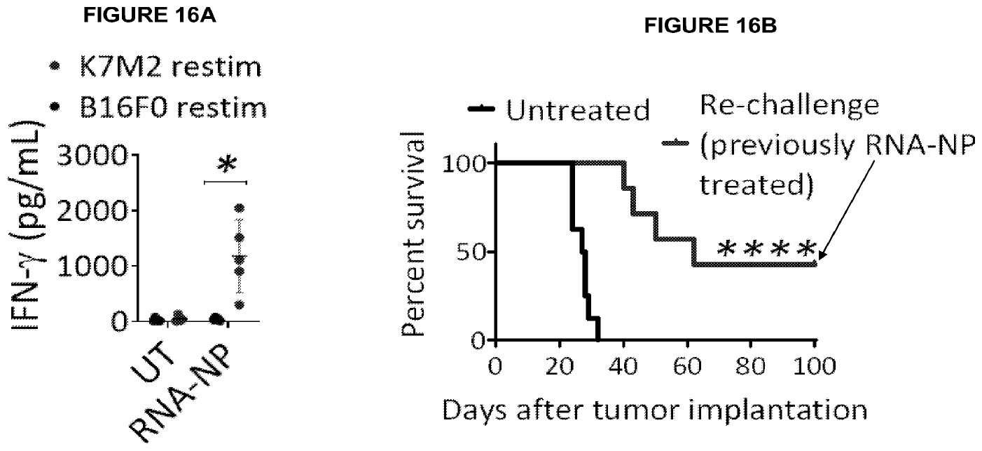

FIG. 18 illustrates that full-length LAMP conjugated pp65 appears to induce greater percentage of antigen specific T cells. Full length LAMP conjugated RNA for pp65 was i.v. administered to naïve mice (n=5/group) once weekly (×3) and spleens were harvested for re-stimulation with overlapping pp65 peptide pool (*p<0.05, Mann-Whitney test). The graph compares IFN-γ production in subjects administered NP alone, RNA-NP, or LAMP RNA-NP.

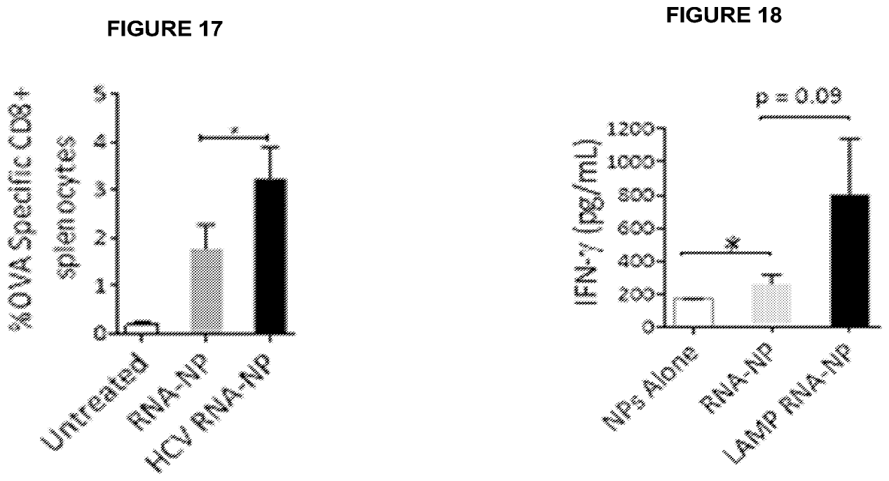

FIGS. 19A and 19B are graphs illustrating % OVA specific Tetramer+ CD8 cells in subjects administered NP alone and RNA-NP in MDAS knock-out subjects. FIG. 19A—T cells alone; FIG. 19B—following restimulation assay with B16F10-OVA. While not wishing to be bound by any particular theory, immunogenicity of multi-lamellar RNA-NPs appears to be dependent on intracellular pathogen recognition receptors such as MDA-5, in contrast to existing mRNA nanolipid platforms.

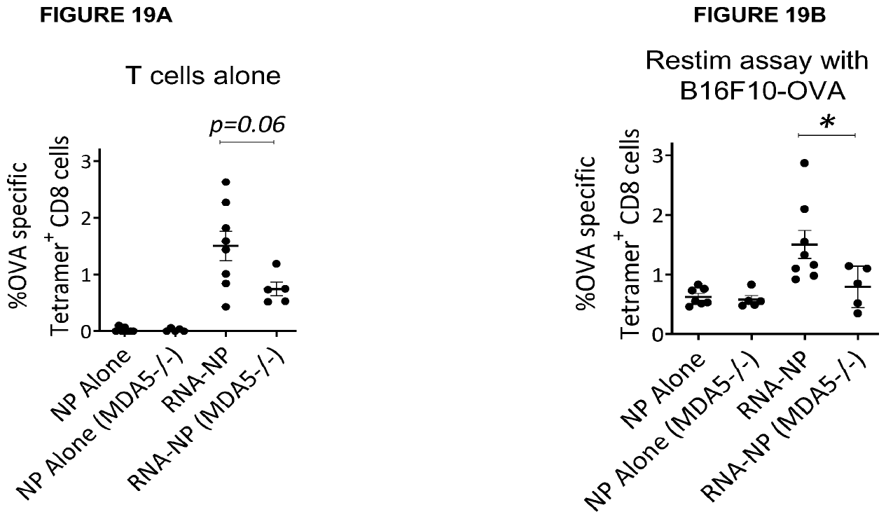

### DETAILED DESCRIPTION

The disclosure provides a method of treating cancer in a human subject, the method comprising administering to the human subject an initial set of doses of nanoparticles every two weeks for an initial treatment period followed by a subsequent set of doses of nanoparticles once a month for a subsequent treatment period. The nanoparticles comprise a positively-charged surface and an interior comprising (i) a core and (ii) at least two nucleic acid layers, each nucleic acid layer being positioned between a cationic lipid bilayer. The nucleic acids (e.g., mRNA) are derived from a cancer cell, and the dose comprises about 0.00050 mg/kg to about 1.5 mg/kg of nucleic acid. Various aspects of the method of the disclosure are described below.

### Nanoparticles

The present disclosure relates to methods of using nanoparticles comprising a cationic lipid and nucleic acids. As used herein the term “nanoparticle” refers to a particle that is less than about 1000 nm in diameter. As the nanoparticles of the present disclosure comprise cationic lipids that have been processed to induce liposome formation, the presently disclosed nanoparticles in various aspects comprise liposomes. Liposomes are artificially-prepared vesicles which, in exemplary aspects, are primarily composed of a lipid bilayer. In various aspects the liposomes of the present disclosure are of different sizes and the composition may comprise one or more of (a) a multilamellar vesicle (MLV) which may be hundreds of nanometers in diameter and may contain a series of concentric bilayers separated by narrow aqueous compartments, (b) a small unicellular vesicle (SUV) which may be smaller than 50 nm in diameter, and (c) a large unilamellar vesicle (LUV) which may be between 50 and 500 nm in diameter.

In exemplary embodiments, the nanoparticles comprise a surface and an interior comprising (i) a core and (ii) at least two nucleic acid layers, optionally, more than two nucleic acid layers. In exemplary instances, each nucleic acid layer is positioned between a lipid layer, e.g., a cationic lipid layer. In exemplary aspects, the nanoparticles are multilamellar comprising alternating layers of nucleic acid and lipid. In exemplary embodiments, the nanoparticle of the present disclosure comprises an interior comprising alternating nucleic acid layers and cationic lipid bilayers. In exemplary embodiments, the nanoparticles comprise at least three nucleic acid layers, each of which is positioned between a cationic lipid bilayer. In exemplary aspects, the nanoparticles comprise at least four or five nucleic acid layers, each of which is positioned between a cationic lipid bilayer. In exemplary aspects, the nanoparticle comprises at least more than five (e.g., 6, 7, 8, 9, 10, 11, 12, 13, 14, 15, 16, 17, 18, 19, 20, or more) nucleic acid layers, each of which is positioned between a cationic lipid bilayer. As used herein, the term “cationic lipid bilayer” is meant a lipid bilayer comprising, consisting essentially of, or consisting of a cationic lipid or a mixture thereof. Suitable cationic lipids are described herein. As used herein the term “nucleic acid layer” is meant a layer of the presently disclosed nanoparticle comprising, consisting essentially of, or consisting of a nucleic acid, e.g., RNA.

In various aspects, the presently disclosed nanoparticle comprises a positively-charged surface. In some instances, the positively-charged surface comprises a lipid layer, e.g., a cationic lipid layer. In various aspects, the outermost layer of the nanoparticle comprises a cationic lipid bilayer. In various instances, the surface comprises a plurality of hydrophilic moieties of the cationic lipid of the cationic lipid bilayer. In some aspects, the core comprises a cationic lipid bilayer. In various instances, the core lacks nucleic acids, optionally, the core comprises less than about 0.5 wt % nucleic acid.

In exemplary aspects, the nanoparticle has a diameter within the nanometer range and accordingly in certain instances are referred to herein as “nanoliposomes” or “liposomes.” In exemplary aspects, the nanoparticle has a diameter between about 50 nm to about 500 nm, e.g., about 50 nm to about 450 nm, about 50 nm to about 400 nm, about 50 nm to about 350 nm, about 50 nm to about 300 nm, about 50 nm to about 250 nm, about 50 nm to about 200 nm, about 50 nm to about 150 nm, about 50 nm to about 100 nm, about 100 nm to about 500 nm, about 150 nm to about 500 nm, about 200 nm to about 500 nm, about 250 nm to about 500 nm, about 300 nm to about 500 nm, about 350 nm to about 500 nm, or about 400 nm to about 500 nm. In exemplary aspects, the nanoparticle has a diameter between about 50 nm to about 300 nm, e.g., about 100 nm to about 250 nm, about 110 nm±5 nm, about 115 nm±5 nm, about 120 nm±5 nm, about 125 nm±5 nm, about 130 nm±5 nm, about 135 nm±5 nm, about 140 nm±5 nm, about 145 nm±5 nm, about 150 nm±5 nm, about 155 nm±5 nm, about 160 nm±5 nm, about 165 nm±5 nm, about 170 nm±5 nm, about 175 nm±5 nm, about 180 nm±5 nm, about 190 nm±5 nm, about 200 nm±5 nm, about 210 nm±5 nm, about 220 nm±5 nm, about 230 nm±5 nm, about 240 nm±5 nm, about 250 nm±5 nm, about 260 nm±5 nm, about 270 nm±5 nm, about 280 nm±5 nm, about 290 nm±5 nm, or about 300 nm±5 nm. In exemplary aspects, the nanoparticle is about 50 nm to about 250 nm in diameter. In some aspects, the nanoparticle is about 70 nm to about 200 nm in diameter.

In exemplary aspects, the nanoparticle is present in a pharmaceutical composition comprising a heterogeneous mixture of nanoparticles ranging in diameter, e.g., about 50 nm to about 500 nm or about 50 nm to about 250 nm in diameter. Optionally, the pharmaceutical composition comprises a heterogeneous mixture of nanoparticles ranging from about 70 nm to about 200 nm in diameter.

In exemplary instances, the nanoparticle is characterized by a zeta potential of about +30 mV to about +60 mV, e.g., about +35 mV to about +55 mV, about +40 mV to about +50 mV, about +45 mV to about +50 mV, about +40 mV to about +45 mV, about +45 mV to about +60 mV, about +50 mV to about +60 mV, or about +55 mV to about +60 mV. In exemplary aspects, the nanoparticle has a zeta potential of about +45 mV to about +55 mV. The nanoparticle in various instances has a zeta potential of about +50 mV. In various aspects, the zeta potential is greater than +30 mV or +35 mV. The zeta potential is one parameter which distinguishes the nanoparticles of the present disclosure and those described in Sayour et al., Oncoimmunology 6(1):e1256527 (2016).

In exemplary embodiments, the nanoparticles comprise a cationic lipid. In some embodiments, the cationic lipid is a low molecular weight cationic lipid such as those described in U.S. Patent Application No. 20130090372, the contents of which are herein incorporated by reference in their entirety. The cationic lipid in exemplary instances is a cationic fatty acid, a cationic glycerolipid, a cationic glycerophospholipid, a cationic sphingolipid, a cationic sterol lipid, a cationic prenol lipid, a cationic saccharolipid, or a cationic polyketide. In exemplary aspects, the cationic lipid comprises two fatty acyl chains, each chain of which is independently saturated or unsaturated. In some instances, the cationic lipid is a diglyceride. For example, in some instances, the cationic lipid may be a cationic lipid of Formula I or Formula II:

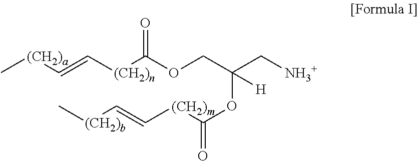

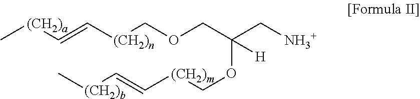

wherein each of a, b, n, and m is independently an integer between 2 and 12 (e.g., between 3 and 10). In some aspects, the cationic lipid is a cationic lipid of Formula I wherein each of a, b, n, and m is independently an integer selected from 3, 4, 5, 6, 7, 8, 9, and 10. In exemplary instances, the cationic lipid is DOTAP (1,2-dioleoyl-3-trimethylammonium-propane), or a derivative thereof. In exemplary instances, the cationic lipid is DOTMA (1,2-di-O-octadecenyl-3-trimethylammonium propane), or a derivative thereof.

In some embodiments, the nanoparticles comprise liposomes formed from 1,2-dioleyloxy-N, N-dimethylaminopropane (DODMA) liposomes, DiLa2 liposomes (e.g., from Marina Biotech (Bothell, Wash.)), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (see, e.g., US20100324120; herein incorporated by reference in its entirety). In some embodiments, the nanoparticles comprise liposomes formed from the synthesis of stabilized plasmid-lipid particles (SPLP) or stabilized nucleic acid lipid particle (SNALP) that have been previously described and shown to be suitable for oligonucleotide delivery in vitro and in vivo. The nanoparticles in some aspects are composed of 3 to 4 lipid components in addition to the nucleic acid molecules. In exemplary aspects, the liposome comprises 55% cholesterol, 20% disteroylphosphatidyl choline (DSPC), 10% PEG-S-DSG, and 15%1,2-dioleyloxy-N,N-dimethylaminopropane (DODMA). In exemplary instances, the liposome comprises 48% cholesterol, 20% DSPC, 2% PEG-c-DMA, and 30% cationic lipid, where the cationic lipid can be 1,2-distearloxy-N, N-dimethylaminopropane (DSDMA), DODMA, DLin-DMA, or 1,2-dilinolenyloxy-3-dimethylaminopropane (DLenDMA).

In some embodiments, the liposomes comprise from about 25.0% cholesterol to about 40.0% cholesterol, from about 30.0% cholesterol to about 45.0% cholesterol, from about 35.0% cholesterol to about 50.0% cholesterol, and/or from about 48.5% cholesterol to about 60% cholesterol. In some embodiments, the liposomes may comprise a percentage of cholesterol selected from the group consisting of 28.5%, 31.5%, 33.5%, 36.5%, 37.0%, 38.5%, 39.0%, and 43.5%. In some embodiments, the liposomes may comprise from about 5.0% to about 10.0% DSPC and/or from about 7.0% to about 15.0% DSPC.

In some embodiments, the liposomes are DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12) 1708-1713); herein incorporated by reference in its entirety) and hyaluronan-coated liposomes (Quiet Therapeutics, Israel).

In various instances, the cationic lipid comprises 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

The liposome in various aspects comprises DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some aspects, the liposome comprises a cationic lipid such as, but not limited to, DLin-DMA, DLin-D-DMA, DLin-MC3-DMA, DLin-KC2-DMA, DODMA and amino alcohol lipids. The amino alcohol cationic lipid comprises in some aspects lipids described in and/or made by the methods described in U.S. Patent Publication No. US 20130150625, herein incorporated by reference in its entirety. As a non-limiting example, the cationic lipid in certain aspects is 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in U.S. 20130150625); 2-amino-3-[(9Z)-octadec-9-en-1-yloxy]-2-{[(9Z)-octadec-9-en-1-yloxy]methyl}propan-1-ol (Compound 2 in US 20130150625); 2-amino-3-[(9Z,12Z)-octadeca-9,12-dien-1-yloxy]-2-[(octyloxy)methyl]propan-1-ol (Compound 3 in US 20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US 20130150625); or any pharmaceutically acceptable salt or stereoisomer thereof.

In various embodiments, the liposome comprises (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-CDMA, in a molar ratio of about 20-60% cationic lipid: 5-25% neutral lipid: 25-55% sterol; 0.5-15% PEG-lipid.

In some embodiments, the liposome comprises from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about 35 to about 65%, from about 45 to about 65%, about 60%, about 57.5%, about 50% or about 40% on a molar basis.

In some embodiments, the liposome comprises from about 0.5% to about 15% on a molar basis of the neutral lipid e.g., from about 3 to about 12%, from about 5 to about 10% or about 15%, about 10%, or about 7.5% on a molar basis. Examples of neutral lipids include, but are not limited to, DSPC, POPC, DPPC, DOPE and SM. In some embodiments, the formulation includes from about 5% to about 50% on a molar basis of the sterol (e.g., about 15 to about 45%, about 20 to about 40%, about 40%, about 38.5%, about 35%, or about 31% on a molar basis). An exemplary sterol is cholesterol. In some embodiments, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis). In some embodiments, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Examples of PEG-modified lipids include, but are not limited to, PEG-distearoyl glycerol (PEG-DMG) (also referred herein as PEG-C14 or C14-PEG), PEG-CDMA (further discussed in Reyes et al. J. Controlled Release, 107, 276-287 (2005) the contents of which are herein incorporated by reference in their entirety).

In exemplary aspects, the cationic lipid may be selected from (20Z,23Z)—N,N-dimethylnonacosa-20,23-dien-10-amine, (17Z,20Z)—N,N-dimemylhexacosa-17,20-dien-9-amine, (1Z,19Z)—N,N-dimethylpentacosa-16,19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N, N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21 Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21 Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, (13Z,16Z)—N,N-dimethyl-3-nonyldocosa-13,16-dien-1-amine, N,N-dimethyl-1-[(1S,2R)-2-octylcyclopropyl]eptadecan-8-amine, 1-[(1S,2R)-2-hexylcyclopropyl]-N, N-dimethylnonadecan-10-amine, N,N-dimethyl-1-[(1S,2R)-2-octylcyclopropyl]nonadecan-10-amine, N, N-dimethyl-21-[(1S,2R)-2-octylcyclopropyl]henicosan-10-amine, N,N-dimethyl-1-[(1S,2S)-2-{[(1R,2R)-2-pentylcyclopropyl]methyl}cyclopropyl]nonadecan-10-amine, N, N-dimethyl-1-[(1S,2R)-2-octylcyclopropyl]hexadecan-8-amine, N, N-dimethyl-[(1R,2S)-2-undecylcyclopropyl]tetradecan-5-amine, N, N-dimethyl-3-{7-[(1S,2R)-2-octylcyclopropyl]heptyl}dodecan-1-amine, 1-[(1R,2S)-2-heptylcyclopropyl]-N, N-dimethyloctadecan-9-amine, 1-[(1S,2R)-2-decylcyclopropyl]-N, N-dimethylpentadecan-6-amine, N, N-dimethyl-1-[(1S,2R)-2-octylcyclopropyl]pentadecan-8-amine, R—N, N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N, N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N, N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N, N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N, N-dimethylpropan-2-amine, 1-[(13Z)-docos-13-en-1-yloxy]-N, N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxyl-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)—N,N-dimethylnonacosa-11,20,2-trien-10-amine or a pharmaceutically acceptable salt or stereoisomer thereof.

In some embodiments, the nanoparticle comprises a lipid-polycation complex. The formation of the lipid-polycation complex may be accomplished by methods known in the art and/or as described in U.S. Patent Publication No. 20120178702, herein incorporated by reference in its entirety. As a non-limiting example, the polycation may include a cationic peptide or a polypeptide such as, but not limited to, polylysine, polyornithine and/or polyarginine. In some embodiments, the composition may comprise a lipid-polycation complex, which may further include a non-cationic lipid such as, but not limited to, cholesterol or dioleoyl phosphatidylethanolamine (DOPE).

In various aspects, the cationic liposomes optionally do not comprise a non-cationic lipid. Neutral molecules, in some aspects, may interfere with coiling/condensation of multi-lamellar NPs resulting in RNA/DNA loaded liposomes greater than 200 nm in size. Cationic liposomes generated without helper molecules can comprise a size of about 70-200 nm (or less). These constructs consist of a cationic lipid with negatively charged nucleic acid, and may be formulated, e.g., in a sealed rotary vacuum evaporator which prevents oxidation of the particles (when exposed to the ambient environment). In this embodiment, the absence of a helper lipid optimizes mRNA coiling into tightly packaged multilamellar NPs where each NP contains a greater amount of nucleic acid per particle. Due to increased nucleic acid payload per particle, these multi-lamellar RNA-NPs drive significantly greater innate immune responses, which are a significant predictor of anti-tumor efficacy.

In some aspects, the nucleic acid molecules are present at a nucleic acid molecule:cationic lipid ratio of about 1 to about 5 to about 1 to about 25, such as about 1 to about 5 to about 1 to about 20, optionally, about 1 to about 18, about 1 to about 17, about 1 to about 15, about 1 to about 10, or about 1 to about 7.5. As used herein, the term “nucleic acid molecule:cationic lipid ratio” is meant a mass ratio, where the mass of the nucleic acid molecule is relative to the mass of the cationic lipid. Also, in exemplary aspects, the term “nucleic acid molecule:cationic lipid ratio” is meant the ratio of the mass of the nucleic acid molecule, e.g., RNA, added to the liposomes comprising cationic lipids during the process of manufacturing the ML RNA NPs of the present disclosure. In exemplary aspects, the nanoparticle comprises less than or about 10 μg RNA molecules per 150 μg lipid mixture. In exemplary aspects, the nanoparticle is made by incubating about 10 μg RNA with about 150 μg liposomes. In alternative aspects, the nanoparticle comprises more RNA molecules per mass of lipid mixture. For example, the nanoparticle may comprise more than 10 μg RNA molecules per 150 μg liposomes. The nanoparticle in some instances comprises more than 15 μg RNA molecules per 150 μg liposomes or lipid mixture.

In various aspects, the nucleic acid molecules are RNA molecules, e.g., transfer RNA (RNA), ribosomal RNA (rRNA), and/or messenger RNA (mRNA). In various aspects, the RNA molecules comprise tRNA, rRNA, mRNA, or a combination thereof. In various aspects, the RNA is total RNA isolated from a cell such as a cell from the subject to be treated. In exemplary aspects, the RNA is total RNA isolated from a diseased cell, such as, for example, a cancer (tumor) cell. Nucleic acid isolated from a diseased cell, such as a cancer cell, is “derived from” the cell. Methods of obtaining total tumor RNA is known in the art and described herein at Example 1.

In exemplary instances, the RNA molecules are mRNA. In various aspects, mRNA is in vitro transcribed mRNA. In various instances, the mRNA molecules are produced by in vitro transcription (IVT). Suitable techniques of carrying out IVT are known in the art. In exemplary aspects, the RNA is in vitro transcribed mRNA, wherein the in vitro transcription template is cDNA made from RNA extracted from a cancer (e.g., tumor) cell. In vitro transcribed mRNA based on DNA sequences from a diseased cell, such as a cancer cell, is “derived from” the cell.

In various aspects, the RNA comprises a sequence encoding a poly(A) tail so that the in vitro transcribed RNA molecule comprises a poly(A) tail at the 3′ end. In various aspects, the method of making a nanoparticle comprises additional processing steps, such as, for example, capping the in vitro transcribed RNA molecules.

The mRNAs in exemplary aspects encode a protein, preferably a tumor antigen. In exemplary aspects, the tumor antigen is an antigen derived from a viral protein, an antigen derived from point mutations, or an antigen encoded by a cancer-germline gene. In exemplary aspects, the tumor antigen is pp65, p53, KRAS, NRAS, MAGEA, MAGEB, MAGEC, BAGE, GAGE, LAGE/NY-ESO1, SSX, tyrosinase, gp100/pmel17, Melan-A/MART-1, gp75/TRP1, TRP2, CEA, RAGE-1, HER2/NEU, WT1. In some aspects, the protein is non-specific relative to a tumor or cancer. For example, the non-specific protein may be green fluorescence protein (GFP) or ovalbumin (OVA).

In exemplary embodiments, the NPs of the present disclosure comprise a mixture of RNA molecules. In exemplary aspects, the mixture of RNA molecules is RNA isolated from cancer cells (e.g., tumor cells) from a human. Optionally, the tumor from which RNA is isolated is selected from any of the cancer types described herein, e.g., selected from the group consisting of a glioma, (including, but not limited to, a glioblastoma), a medulloblastoma, a diffuse intrinsic pontine glioma, and a peripheral tumor with metastatic infiltration into the central nervous system (e.g., melanoma or breast cancer). The nucleic acid may be derived from a cancer cell from the subject or from a cancer cell originating in a different individual.

In various aspects, the nucleic acid molecule (e.g., RNA molecule) further comprises a nucleotide sequence encoding a lysosome-associated membrane protein (LAMP). LAMPs are membrane proteins specific to lysosomes comprising homologous lysosome-luminal domains separated by a proline-rich hinge region, a transmembrane domain and a cytoplasmic domain. A review on LAMPs is provided at Schwake et al., Traffic (2013) koi.org/10.1111/tra.12056. In certain aspects, the LAMP protein is a LAMP1, LAMP 2, LAMP3, LAMP4, or LAMP5 protein. The sequences of such LAMP proteins are known in the art. For example, the mRNA sequence of the LAMP1 precursor is available as NCBI Accession No. NM_005561.4 and the amino acid sequence of LAMP1 precursor is available as NCBI Accession No. NP_005552.3. Also, for example, the mRNA sequence of the LAMP2 isoform C precursor is available as NCBI Accession No. NM_001122606.1 and the amino acid sequence of LAMP2 isoform C precursor is available as NCBI Accession No. NP_001116078.1. The mRNA sequence of the LAMP3 precursor is available as NCBI Accession No. NM_014398.4 and the amino acid sequence of LAMP3 precursor is available as NCBI Accession No. NP_055213.2. The disclosures of the aforementioned accession numbers are hereby incorporated by reference.

In various aspects, the NP of the present disclosure comprises autologous total tumor mRNA and, optionally, pp65 full length lysosomal associated membrane protein (LAMP) mRNA loaded into a nanoparticle described herein comprising the cationic lipid DOTAP (1,2-dioleoyl-3-trimethylammonium-propane).

In various instances, the mRNA comprises a nucleotide sequence encoding an amino acid sequence set forth in the Sequence Listing. In various aspects, the nucleotide sequence encodes an amino acid sequence which has at least 50%, at least 60%, at least 70%, at least 80%, at least 85%, at least 90% or has greater than 90% sequence identity (e.g., about 91%, about 92%, about 93%, about 94%, about 95%, about 96%, about 97%, about 98%, about 99%) to an amino acid sequence set forth in the Sequence Listing. In various aspects, the nucleotide sequence encodes an amino acid sequence set forth in the Sequence Listing with 1 to 10 amino acid substitutions, e.g., about 1 to 9, about 1 to 8, about 1 to 7, about 1 to 6, about 1 to 5, about 1 to 4, about 1 to 3, about 1 to 2 amino acid substitutions or 1, 2, 3, 4, 5, 6, 7, 8, 9, 10 amino acid substitutions.

Nanoparticles are further described in, e.g., International Patent Application No. PCT/US20/42606, which is hereby incorporated by reference in its entirety and in particular with respect to the discussion of nanoparticles comprising a positively-charged surface and an interior comprising a core and at least two nucleic acid layers, wherein each nucleic acid layer is positioned between a cationic lipid bilayer, and methods of manufacture.

### Cancer Treatment and Related Methods

The data provided herein support the use of the presently disclosed RNA NPs for inducing or increasing an immune response against a cancer (e.g., a tumor) in a subject. Accordingly, a method of inducing or increasing an immune response against a cancer (e.g., a tumor) in a subject is provided by the present disclosure.

The disclosure provides a method of treating cancer in a human subject, the method comprising (a) administering to the human subject a dose of nanoparticles every two weeks for an initial treatment period, then (b) administering a dose of nanoparticles once a month for a subsequent treatment period, wherein each dose comprises about 0.00050 mg/kg to about 1.5 mg/kg of nucleic acid. The nucleic acid may be encapsulated into any suitable amount of liposome to administer the nucleic acid to the subject. As explained above, in various embodiments, the nucleic acid molecules may be present in the nanoparticle at a nucleic acid molecule:cationic lipid ratio of about 1 to about 5 to about 1 to about 25, such as about 1 to about 5 to about 1 to about 20, optionally, about 1 to about 18, about 1 to about 17, about 1 to about 15, about 1 to about 10, or about 1 to about 7.5. In various aspects, the amount of nucleic acid for administration is encapsulated into about 0.008 mg/kg to about 1.5 mg/kg of liposome material (“LP”). In various aspects, the dose of nanoparticles comprises about 0.000625 mg/kg to about 0.08 mg/kg nucleic acid (e.g., RNA, such as mRNA), which is optionally encapsulated in about 0.009375 mg/kg to about 1.2 mg/kg LPs. Examples of doses suitable for use in the context of the disclosure include, but are not limited to about 0.000625 mg/kg of nucleic acid (e.g., mRNA) optionally encapsulated in about 0.009375 mg/kg LPs, about 0.00125 mg/kg nucleic acid (e.g., mRNA) optionally encapsulated in about 0.01875 mg/kg LPs, about 0.0025 mg/kg nucleic acid (e.g., mRNA) optionally encapsulated in about 0.0375 mg/kg LPs, about 0.005 mg/kg nucleic acid (e.g., mRNA) optionally encapsulated in about 0.075 mg/kg LPs, about 0.01 mg/kg nucleic acid (e.g., mRNA) optionally encapsulated in 0.15 mg/kg LPs, about 0.02 mg/kg nucleic acid (e.g., mRNA) optionally encapsulated in about 0.3 mg/kg LPs, about 0.04 mg/kg nucleic acid (e.g., mRNA) optionally encapsulated in about 0.6 mg/kg LPs, and about 0.08 mg/kg nucleic acid (e.g., mRNA) optionally encapsulated in 1.2 mg/kg LPs.

In various aspects, the dose(s) of nanoparticles administered is effective to activate dendritic cells (DCs) in the subject. In various instances, the dose(s) of nanoparticles administered is effective to induce a T cell-mediated immune response, for instance, a CD8+ T cell-mediated immune response. Optionally, the T cell-mediated immune response comprises an increase in effector memory T cells specific to cancer antigen (e.g., tumor protein). In various aspects, the T-cell mediated immune response comprises an increase in CD3+CD8+CD44+ cells. Optionally, the T cell-mediated immune response comprises activity by tumor infiltrating lymphocytes (TILs). In various instances, the dose(s) of nanoparticles administered is effective to induce a B-cell-mediated immune response, e.g., involving the production of antibodies (e.g., neutralizing antibodies). In various instances, the immune response involves an increase in the production of antibodies specific to a cancer antigen (e.g., a tumor protein), optionally, IgM and/or IgG antibodies. In various instances, the dose(s) of nanoparticles administered is effective to induce the innate immune response. Optionally, the immune response is an innate immune response involving one or more of granulocytes, monocytes, macrophages, and natural killer (NK) cells. In various aspects, the presently disclosed multilamellar RNA NPs are administered to a subject for inducing or increasing an immune response against a cancer antigen (e.g., tumor protein) in a subject, wherein DCs are activated, B-cells produce neutralizing antibodies to the cancer, the number of effector memory T-cells specific to the cancer is increased, the innate immune response against the cancer is activated, or a combination thereof.

The doses of nanoparticles may be administered to a subject over the course of a treatment period (i.e., a “total treatment period”) of about 18 months or less, about 16 months or less, about 14 months or less, or about one year (12 months) or less (e.g., about nine months or less, about six months or less, or about three months or less).

The treatment regimen includes more frequent administration of nanoparticles over an initial treatment period, then subsequent administrations spaced out over a longer period of time. For example, doses may be administered for an initial treatment period of about two weeks, about four weeks, about six weeks, about eight weeks, about 10 weeks, or about 12 weeks, during which multiple doses are administered at an interval of, e.g., once a week or every two weeks. The treatment regimen then comprises a subsequent treatment period wherein multiple doses are administered with longer intervals between doses, e.g., every two weeks, every three weeks, every four weeks (i.e. once a month), every five weeks, every six weeks, and the like. The subsequent treatment period may comprise about four weeks, about five weeks, about six weeks, about seven weeks, about eight weeks, about three months, about four months, about five months, about six months, about seven months, about eight months, about nine months, about 10 months, about 11 months, about 12 months, or more. The length of the initial treatment period and the subsequent treatment period may, or may not, be the same (the interval between administrations within the initial treatment period and subsequent treatment period will differ).

In an exemplary aspect of the disclosure, a dose of nanoparticles is administered about every two weeks for an initial treatment period of about four weeks (totaling three initial doses), which is followed by administration of a dose of nanoparticles about once a month for a period of about twelve months. The initial treatment period and the subsequent treatment period total about 18 months, in various embodiments.

As used herein, the term “treat,” as well as words related thereto, do not necessarily imply 100% or complete treatment (i.e., complete remission or eradication of the disease). The methods of treating a disease of the present disclosure can provide any amount or any level of treatment. For example, a therapeutic response optionally refers to one or more of the following improvements in the disease: (1) a reduction in the number of neoplastic cells; (2) an increase in neoplastic cell death; (3) inhibition of neoplastic cell survival; (4) inhibition (i.e., slowing to some extent, preferably halting) of tumor growth or appearance of new lesions; (5) an increased patient survival rate; and/or (6) some relief from one or more symptoms associated with the disease or condition (e.g., pain, weight loss, weakness or fatigue, anemia, or bleeding). Disease state is monitored by, e.g., clinical examination, X-ray, computerized tomography (CT, such as spiral CT), magnetic resonance imaging (MRI), positron emission tomography (PET), ultrasound, endoscopy and laparoscopy, tumor marker levels (e.g., carcinoembryonic antigen (CEA)), cytology, histology, tumor biopsy sampling, and/or counting of tumor cells in circulation. The treatment provided by the presently disclosed method may delay the onset or reoccurrence/relapse of the disease being prophylactically treated. The prophylactic treatment encompasses reducing the risk of the disease being treated. In exemplary aspects, the method reduces the risk of the disease or relapse by 2-fold, 5-fold, 10-fold, 20-fold, 50-fold, 100-fold, or more.

In various aspects, response to treatment is evaluated by measuring parameters of one or more target lesions over time. For glioma, for instance, response determination may be based on a comparison of an area [W (longest diameter of the target lesion)×T (transverse measurement, perpendicular to W)] between the baseline assessment and after treatment. A complete response is characterized by the disappearance of lesions. A partial response is characterized by at least a 50% decrease in the size of target lesions. A subject exhibiting “stable disease” exhibits neither sufficient shrinkage to qualify for complete response or partial response nor sufficient increase to qualify for progressive disease, characterized by at least a 25% increase in the sum of the size of target lesions. The disclosure contemplates improvement of any of these parameters, and preferably improvement sufficient to achieve at least a partial response. For example, in various aspects, the subject achieves at least a 10% reduction, at least a 20% reduction, at least a 30% reduction (e.g., at least a 40% reduction, at least a 50% reduction, at least a 60% reduction, at least a 70% reduction, at least an 80% reduction, or at least a 90% reduction) in the area of target lesions (compared to baseline before treatment) or demonstrates a complete response. Alternatively or in addition, the subject experiences progression-free survival for at least six months (e.g., at least nine months) after cessation of treatment, optionally experiencing progression-free survival for 12 months or longer (e.g., 18 months or longer or 24 months or longer) after cessation of treatment. The method of the disclosure may also improve the stage or grade of the cancer.

The cancer treatable by the methods disclosed herein may be any cancer, e.g., any malignant growth or tumor caused by abnormal and uncontrolled cell division that may spread to other parts of the body through the lymphatic system or the blood stream (although this is not required). In exemplary aspects, the cancer is located across the blood brain barrier and/or the subject has a tumor located in the brain. In some aspects, the tumor is a glioma, a low grade glioma or a high grade glioma, specifically a grade III astrocytoma or a glioblastoma. Alternatively, the tumor could be a medulloblastoma or a diffuse intrinsic pontine glioma. In another example, the tumor could be a metastatic infiltration from a non-CNS tumor, e.g., breast cancer, melanoma, or lung cancer.

The cancer in some aspects is one selected from the group consisting of acute lymphocytic cancer, leukemia (e.g., acute lymphoblastic leukemia (ALL), acute myeloid leukemia (AML), chronic lymphocytic leukemia (CLL), or chronic myeloid leukemia (CML)), alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic myeloid cancer, colon cancer, esophageal cancer, cervical cancer, gastrointestinal carcinoid tumor, lymphoma (e.g., Hodgkin lymphoma, non-Hodgkin lymphoma, B-cell lymphoma (DLBCL), follicular lymphoma, mantle cell lymphoma (MCL), or small lymphocytic lymphoma (SLL)), hypopharynx cancer, kidney cancer, larynx cancer, liver cancer, lung cancer, malignant mesothelioma, melanoma, multiple myeloma, nasopharynx cancer, ovarian cancer, pancreatic cancer, peritoneum, omentum, and mesentery cancer, pharynx cancer, prostate cancer, renal cancer (e.g., renal cell carcinoma (RCC)), small intestine cancer, soft tissue cancer, stomach cancer, testicular cancer, thyroid cancer, ureter cancer, and urinary bladder cancer. In particular aspects, the cancer is selected from the group consisting of head and neck, ovarian, cervical, bladder and oesophageal cancers, pancreatic, gastrointestinal cancer, gastric, breast, endometrial and colorectal cancers, hepatocellular carcinoma, glioblastoma, bladder, and lung cancer (e.g., non-small cell lung cancer (NSCLC), small-cell lung cancer (SCLC), or bronchioloalveolar carcinoma).

With regard to the foregoing methods, the NPs or the composition comprising the same in some aspects is systemically administered to the subject. Optionally, the method comprises administration of the liposomes or composition by way of parenteral administration. In various instances, the liposome or composition is administered to the subject intravenously.

### Subjects

The subject is a human. In some aspects, the human is an adult subject aged 21 years or older. In some aspects, the human is a pediatric subject aged less than 21 years (e.g., aged at least three years and less than 21 years). In exemplary aspects, the subject has or is suspected as suffering from a cancer, such as a cancer described here, e.g., glioma. In exemplary aspects, the subject has or is suspected as suffering from glioblastoma (e.g., Glioblastoma multiforme (GBM)). In exemplary aspects the subject has or is suspected as suffering from a diffuse midline glioma (DMG) or diffuse intrinsic pontine glioma (DIPG). Alternatively, the subject may have or be suspected as suffering from Newly Diagnosed Pediatric High-Grade Gliomas (pHGG). Optionally, the gliobastoma is MGMT unmethylated. Optionally, the subject has undergone or will undergo surgical resection of suspected glioma (e.g., GBM or HGG).

### Pharmaceutical Compositions

The nanoparticle is typically provided in the form of a pharmaceutical composition comprising a plurality of nanoparticles according to the present disclosure and a pharmaceutically acceptable carrier, diluent, or excipient and intended for administration to a human. In exemplary aspects, the composition is a sterile composition. Optionally, at least 50% of the nanoparticles of the plurality have a diameter between about 100 nm to about 250 nm. In various aspects, the composition comprises about 10¹⁰ nanoparticles per mL to about 10¹⁵ nanoparticles per mL, optionally about 10¹² nanoparticles±10% per mL.

In exemplary aspects, the composition of the present disclosure may comprise additional components other than the nanoparticle. The composition, in various aspects, comprises any pharmaceutically acceptable ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, UK, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

The composition of the present disclosure can be suitable for administration by any acceptable route, including parenteral and subcutaneous routes. Other routes include intravenous, intradermal, intramuscular, intraperitoneal, intranodal and intrasplenic, for example. In exemplary aspects, the composition is suitable for systemic (e.g., intravenous) administration.

If the composition is in a form intended for administration to a subject, it can be made to be isotonic with the intended site of administration. For example, if the solution is in a form intended for administration parenterally, it can be isotonic with blood. The composition typically is sterile. In certain embodiments, this may be accomplished by filtration through sterile filtration membranes. In certain embodiments, parenteral compositions generally are placed into a container having a sterile access port, for example, an intravenous solution bag, or vial having a stopper pierceable by a hypodermic injection needle, or a prefilled syringe. In certain embodiments, the composition may be stored either in a ready-to-use form or in a form (e.g., lyophilized) that is reconstituted or diluted prior to administration.

### Methods of Manufacture

The nanoparticles of the disclosure may be made by (A) mixing nucleic acid molecules and liposomes at a RNA:liposome ratio described herein, e.g., a ratio of about 1 to about 5 to about 1 to about 25, such as about 1 to about 5 to about 1 to about 20, optionally, about 1 to about 15, to obtain a RNA-coated liposomes, wherein the liposomes are made by a process of making liposomes comprising drying a lipid mixture comprising a cationic lipid and an organic solvent by evaporating the organic solvent under a vacuum; and (B) mixing the RNA-coated liposomes with a surplus amount of liposomes.

In exemplary aspects, the nanoparticle made by the presently disclosed method accords with the descriptions of the presently disclosed nanoparticles described herein. For example, the nanoparticle made by the presently disclosed methods has a zeta potential of about +30 mV to about +60 mV, optionally, about +45 mV to about +55 mV. Optionally, the zeta potential of the nanoparticle made by the presently disclosed methods is about +50 mV. In various aspects, the core of the nanoparticle made by the presently disclosed methods comprises less than about 0.5 wt % nucleic acid and/or the core comprises a cationic lipid bilayer and/or the outermost layer of the nanoparticle comprises a cationic lipid bilayer and/or the surface of the nanoparticle comprises a plurality of hydrophilic moieties of the cationic lipid of the cationic lipid bilayer.

In exemplary aspects, the lipid mixture comprises the cationic lipid and the organic solvent at a ratio of about 40 mg cationic lipid per mL organic solvent to about 60 mg cationic lipid per mL organic solvent, optionally, at a ratio of about 50 mg cationic lipid per mL organic solvent. In various instances, the process of making liposomes further comprises rehydrating the lipid mixture with a rehydration solution to form a rehydrated lipid mixture and then agitating, resting, and sizing the rehydrated lipid mixture. Optionally, sizing the rehydrated lipid mixture comprises sonicating, extruding and/or filtering the rehydrated lipid mixture.

A description of an exemplary method of making a nanoparticle comprising a positively-charged surface and an interior comprising (i) a core and (ii) at least two nucleic acid layers, wherein each nucleic acid layer is positioned between a cationic lipid bilayer is provided herein at Example 1. Any one or more of the steps described in Example 1 may be included in the presently disclosed method. For instance, in some embodiments, the method comprises one or more steps required for preparing the RNA prior to being complexed with the liposomes. In exemplary aspects, the method comprises downstream steps to prepare the nanoparticles for administration to a subject, e.g., a human. In exemplary instances, the method comprises formulating the NP for intravenous injection. The method comprises in various aspects adding one or more pharmaceutically acceptable carriers, diluents, or excipients, and optionally comprises packaging the resulting composition in a container, e.g., a vial, a syringe, a bag, an ampoule, and the like. The container in some aspects is a ready-to-use container and optionally is for single-use.

### Imaging Agents

Multifunctional RNA-loaded liposomes initiate potent antitumor immunity and may also be employed to function as an early MRI-based imaging biomarker of treatment response. The nanoparticles, in various aspects, optionally comprise a diagnostic agent, such as an imaging agent (e.g., a contrast agent), optionally, gadolinium, a perfluorocarbon microbubble, iron oxide nanoparticle, colloidal gold or gold nanoparticle (see, e.g., Mahan and Doiron, J Nanomaterials, volume 2018, article ID 5837276). In various aspects, nanoparticle comprises core comprising a radiopharmaceutical (e.g., carbon-11, fluorine-18, gallium-67 or 68, indium-111, iodine-123, -125, -131, krypton-81m, lutetium-177, nitrogen-13, oxygen-15, phosphorus-32, selenium-75, technetium-99m, thallium-201, xenon-133, yttrium-90). In various aspects, the core comprises iron oxide nanoparticles (IONPs) which are useful for imaging tissue or cells via, e.g., magnetic resonance imaging (MRI). In various aspects, the IONPs are Combidex®, Resovist®, Endorem®, or Sinerem®. Optionally, the IONPs are coated with a fatty acid, e.g., a C8-C30 fatty acid. In various aspects, the fatty acid is stearic acid, palmitic acid, myristic acid, lauric acid, capric acid, caprylic acid, palmitoleic acid, cis-vaccenic acid, or oleic acid. In various aspects, the core comprises a plurality of IONPs (optionally wherein each IONP is coated with oleic acid) wherein the plurality is held together by a lipid, e.g., a cationic lipid. Optionally, the plurality of IONPs (optionally coated with oleic acid) are held together by DOTAP.

### Other Therapeutic Agents

The disclosure contemplates a treatment regimen comprising administration of the nanoparticles of the disclosure and administration of one or more other therapies, including chemotherapeutic agents. The one or more other therapies may be provided before, during, or after the course of treatment with the nanoparticles described herein. Optionally, the nanoparticles of the disclosure comprise an additional therapeutic moiety incorporated in the structure of the nanoparticle. Alternatively, additional therapeutic agents are administered separately according to a treatment regimen appropriate for the particular agent.

Chemotherapeutic agents suitable for inclusion in the presently disclosed multilamellar RNA NPs include, but are not limited to, platinum coordination compounds, topoisomerase inhibitors, antibiotics, antimitotic alkaloids and difluoronucleosides, as described in U.S. Pat. No. 6,630,124 (incorporated herein by reference).

In some embodiments, the chemotherapeutic agent is a platinum coordination compound. The term “platinum coordination compound” refers to any tumor cell growth inhibiting compound that provides platinum in the form of an ion. In some embodiments, the platinum coordination compound is cis-diamminediaquoplatinum (II)-ion; chloro(diethylenetriamine)-platinum(II)chloride; dichloro(ethylenediamine)-platinum(II), diammine(1,1-cyclobutanedicarboxylato) platinum(II) (carboplatin); spiroplatin; iproplatin; diammine(2-ethylmalonato)-platinum(II); ethylenediaminemalonatoplatinum(II); aqua(1,2-diaminodyclohexane)-sulfatoplatinum(II); (1,2-diaminocyclohexane)malonatoplatinum(II); (4-caroxyphthalato)(1,2-diaminocyclohexane)platinum(II); (1,2-diaminocyclohexane)-(isocitrato)platinum(II); (1,2-diaminocyclohexane)cis(pyruvato)platinum(II); (1,2-diaminocyclohexane)oxalatoplatinum(II); ormaplatin; or tetraplatin.

In some embodiments, cisplatin is the platinum coordination compound employed in the compositions and methods of the present disclosure. Cisplatin is commercially available under the name PLATINOL™ from Bristol Myers-Squibb Corporation and is available as a powder for constitution with water, sterile saline or other suitable vehicle. Other platinum coordination compounds suitable for use in the context of the present disclosure are known and are available commercially and/or can be prepared by known techniques. Cisplatin, or cis-dichlorodiammineplatinum II, has been used successfully for many years as a chemotherapeutic agent in the treatment of various human solid malignant tumors. More recently, other diamino-platinum complexes have also shown efficacy as chemotherapeutic agents in the treatment of various human solid malignant tumors. Such diamino-platinum complexes include, but are not limited to, spiroplatinum and carboplatinum. Although cisplatin and other diamino-platinum complexes have been widely used as chemotherapeutic agents in humans, they have had to be delivered at high dosage levels that can lead to toxicity problems such as kidney damage.

In some embodiments, a chemotherapeutic agent is provided to the subject which is a topoisomerase inhibitor. Various topoisomerase inhibitors have shown clinical efficacy in the treatment of humans afflicted with ovarian cancer, breast cancer, esophageal cancer or non-small cell lung carcinoma. In some aspects, the topoisomerase inhibitor is camptothecin or a camptothecin analog. Camptothecin is a water-insoluble, cytotoxic alkaloid produced by Camptotheca accuminata trees indigenous to China and Nothapodytes foetidatrees indigenous to India. Camptothecin inhibits growth of a number of tumor cells. Compounds of the camptothecin analog class are typically specific inhibitors of DNA topoisomerase I. Compounds of the camptothecin analog class include, but are not limited to; topotecan, irinotecan and 9-amino-camptothecin.

In additional embodiments, the chemotherapeutic agent is any tumor cell growth inhibiting camptothecin analog claimed or described in: U.S. Pat. No. 5,004,758 and European Patent Application Number 88311366.4, published as EP 0 321 122; U.S. Pat. No. 4,604,463 and European Patent Application Publication Number EP 0 137 145; U.S. Pat. No. 4,473,692 and European Patent Application Publication Number EP 0 074 256; U.S. Pat. No. 4,545,880 and European Patent Application Publication Number EP 0 074 256; European Patent Application Publication Number EP 0 088 642; Wani et al., J. Med. Chem., 29, 2358-2363 (1986); Nitta et al., Proc. 14th International Congr. Chemotherapy, Kyoto, 1985, Tokyo Press, Anticancer Section 1, p. 28-30; especially a compound called CPT-11. CPT-11 is a camptothecin analog with a 4-(piperidino)-piperidine side chain joined through a carbamate linkage at C-10 of 10-hydroxy-7-ethyl camptothecin. CPT-11 is currently undergoing human clinical trials and is also referred to as irinotecan; Wani et al, J. Med. Chem., 23, 554 (1980); Wani et. al., J. Med. Chem., 30, 1774 (1987); U.S. Pat. No. 4,342,776; European Patent Application Publication Number EP 418 099; U.S. Pat. No. 4,513,138 and European Patent Application Publication Number EP 0 074 770; U.S. Pat. No. 4,399,276 and European Patent Application Publication Number 0 056 692; the entire disclosure of each of which is hereby incorporated by reference. All of the above-listed compounds of the camptothecin analog class are available commercially and/or can be prepared by known techniques including those described in the above-listed references. The topoisomerase inhibitor may be selected from the group consisting of topotecan, irinotecan and 9-aminocamptothecin.

In still yet other embodiments, an antibiotic compound is provided to the subject in connection with the method described herein. Suitable antibiotic include, but are not limited to, doxorubicin, mitomycin, bleomycin, daunorubicin, and streptozocin.

In some embodiments, the chemotherapeutic agent is an antimitotic alkaloid. In general, antimitotic alkaloids can be extracted from Catharanthus roseus, and have been shown to be efficacious as anticancer chemotherapy agents. A great number of semi-synthetic derivatives have been studied both chemically and pharmacologically (see, O. Van Tellingen et al, Anticancer Research, 12, 1699-1716 (1992)). Antimitotic alkaloids include, but are not limited to, vinblastine, vincristine, vindesine, paclitaxel (PTX; Taxol®) and vinorelbine. The latter two antimitotic alkaloids are commercially available from Eli Lilly and Company, and Pierre Fabre Laboratories, respectively (see, U.S. Pat. No. 5,620,985). In an exemplary aspect, the antimitotic alkaloid is vinorelbine.

In other embodiments, the chemotherapeutic agent is a difluoronucleoside. 2 [symbol] deoxy-2 [symbol] 2 [symbol] difluoronucleosides are known in the art as having antiviral activity. Such compounds are disclosed in U.S. Pat. Nos. 4,526,988 and 4,808,614. European Patent Application Publication 184,365 discloses that these same difluoronucleosides have oncolytic activity. In certain specific aspects, the 2 [symbol] deoxy-2 [symbol] 2 [symbol] difluoronucleoside used in the compositions and methods of the present invention is 2 [symbol] deoxy-2 [symbol] 2 [symbol] difluorocytidine hydrochloride, also known as gemcitabine hydrochloride. Gemcitabine is commercially available or can be synthesized in a multi-step process as disclosed and taught in U.S. Pat. Nos. 4,526,988, 4,808,614 and 5,223,608, the teachings of which are incorporated herein by reference.

In exemplary aspects, the chemotherapeutic agent is a hormone therapy agent. In exemplary instances, the hormone therapy agent is, for instance, letrozole, tamoxifen, bazedoxifene, exemestane, leuprolide, goserelin, fulvestrant, anastrozole, or toremifene. In exemplary aspects, the hormone therapy agent is a luteinizing hormone (LH) blocker, e.g., gosarelin, or an LH releasing hormone (RH) agonist. In exemplary aspects, the hormone therapy agent is an ER-targeted agent (e.g., fulvestrant or tamoxifen), rapamycin, a rapamycin analog (e.g., everolimus, temsirolimus, ridaforolimus, zotarolimus, and 32-deoxo-rapamycin), an anti-HER2 drug (e.g., trastuzumab, pertuzumab, lapatinib, T-DM1, or neratinib) or a PI3K inhibitor (e.g., taselisib, alpelisib or buparlisib).

The following examples are given merely to illustrate the present invention and not in any way to limit its scope.

### EXAMPLES

### Example 1

This example describes a method of making nanoparticles of the present disclosure.

### Preparation of DOTAP Liposomes

On Day 1, the following steps were carried out in the fume hood. Water was added to a rotavapor bath. Chloroform (20 mL) was poured into a sterile, glass graduated cylinder. After opening a vial containing 1 g of DOTAP, 5 mL chloroform was added to the DOTAP vial using a glass pipette. The volume of chloroform and DOTAP was then transferred into a 1-L evaporating flask. The DOTAP vial was washed by adding a second 5-mL volume of chloroform to the DOTAP vial to dissolve any remaining DOTAP in the vial and then transferring this volume of chloroform from the DOTAP vial to the evaporating flask. This washing step was repeated 2 more times until all the chloroform in the graduated cylinder was used. The evaporating flask was then placed into the Buchi rotavapor. The water bath was turned on and adjusted to 25° C. The evaporating flask was moved downward until it touched the water bath. The rotation speed of the rotavapor was adjusted to 2. The vacuum system was turned on and adjusted to 40 mbar. After 10 minutes, the vacuum system was turned off and the chloroform was collected from the collector flask. The amount of chloroform collected was measured. Once the collector flask is repositioned, the vacuum was turned on again and the contents in the evaporating flask was allowed to dry overnight until the chloroform was completely evaporated.

On Day 2, using a sterile graduated cylinder, PBS (200 mL) was added to a new, sterile 500-mL PBS bottle maintained at room temperature. A second 500-mL PBS bottle was prepared for collecting DOTAP. The Buchi rotavapor water bath was set to 50° C. PBS (50 mL) was added into the evaporating flask using a 25-mL disposable serological pipette. The evaporating flask was positioned in the Buchi rotavapor and moved downward until ⅓ of the flask was submerged into the water bath. The rotation speed of the rotavapor was set to 2, allowed to rotate for 10 min, and then rotation was turned off. A 50-mL volume of PBS with DOTAP from the evaporating flask was transferred to the second 500 mL PBS bottle. The steps were repeated (3-times) until the entire volume of PBS in the PBS bottle was used. The final volume of the second 500 mL PBS bottle was 400 mL. The lipid solution in the second 500 mL PBS bottle was vortexed for 30 s and then incubated at 50° C. for 1 hour. During the 1 hour incubation, the bottle was vortexed every 10 min. The second 500 mL PBS bottle was allowed to rest on overnight at room temperature.

On Day 3, PBS (200 mL) was added to the second 500 mL PBS bottle containing DOTAP and PBS. The second 500 mL PBS bottle was placed into an ultrasonic bath. Water was filled in the ultrasonic bath and the second 500 mL PBS bottle was sonicated for 5 min. The extruder was washed with PBS (100 mL) and this wash step was repeated. A 0.45 μm pore filter was assembled into a filtration unit and a new (third) 500 mL PBS bottle was positioned into the output tube of the extruder. In a biological safety cabinet, the DOTAP-PBS mixture was loaded into the extruder, until about 70% of the third PBS bottle was filled. The extruder was then turned on and the DOTAP PBS mixture was added until all the mixture was run through the extruder. Subsequently, a 0.22 μm pore filter was assembled into the filtration unit and a new (third) 500 mL PBS bottle was positioned into the output tube of the extruder. The previously filtered DOTAP-PBS mixture was loaded and run again throughout. The samples comprising DOTAP lipid nanoparticles (NPs) in PBS were then stored at 4° C.

### RNA Preparation

Prior to incorporation into NPs, RNA was prepared in one of a few ways (A) to (C). Total tumor RNA was prepared by isolating total RNA (including rRNA, tRNA, mRNA) from tumor cells. In vitro transcribed mRNA was prepared by carrying out in vitro transcription reactions using cDNA templates produced by reverse transcription of total tumor RNA. Tumor antigen-specific and non-specific RNAs were either made in-house or purchased from a vendor.

### (A) Total Tumor RNA:

Total tumor-derived RNA from tumor cells (e.g., B16F0, B16F10, and KR158-luc) is isolated using commercially available RNeasy mini kits (Qiagen) based on manufacturer instructions.

(B) In Vitro Transcribed mRNA:

Briefly, RNA is isolated using commercially available RNeasy mini kits (Qiagen) per manufacturer's instructions and cDNA libraries were generated by RT-PCR. Using a SMARTScribe Reverse Transcriptase kit (Takara), a reverse transcriptase reaction by PCR was performed on the total tumor RNA in order to generate cDNA libraries. The resulting cDNA was then amplified using Takara Advantage 2 Polymerase mix with T7/SMART and CDS III primers, with the total number of amplification cycles determined by gel electrophoresis. Purification of the cDNA was performed using a Qiagen PCR purification kit per manufacturer's instructions. In order to isolate sufficient mRNA for use in each RNA-nanoparticle vaccine, mMESAGE mMACHINE (Invitrogen) kits with T7 enzyme mix were used to perform overnight in vitro transcription on the cDNA libraries. Housekeeping genes were assessed to ensure fidelity of transcription. The resulting mRNA was then purified with a Qiagen RNeasy Maxi kit to obtain the final mRNA product.

(C) Tumor Antigen-Specific and Non-Specific mRNA:

Plasmids comprising DNA encoding tumor antigen-specific RNA (RNA encoding, e.g., pp65, OVA) and non-specific RNA (RNA encoding, e.g., Green Fluorescent Protein (GFP), luciferase) are linearized using restriction enzymes (i.e., SpeI) and purified with Qiagen PCR MiniElute kits. Linearized DNA is subsequently transcribed using the mmRNA in vitro transcription kit (Life technologies, Invitrogen) and cleaned up using RNA Maxi kits (Qiagen). In alternative methods, non-specific RNA is purchased from Trilink Biotechnologies (San Diego, CA).

### Preparation of Multilamellar RNA Nanoparticles (NPs)

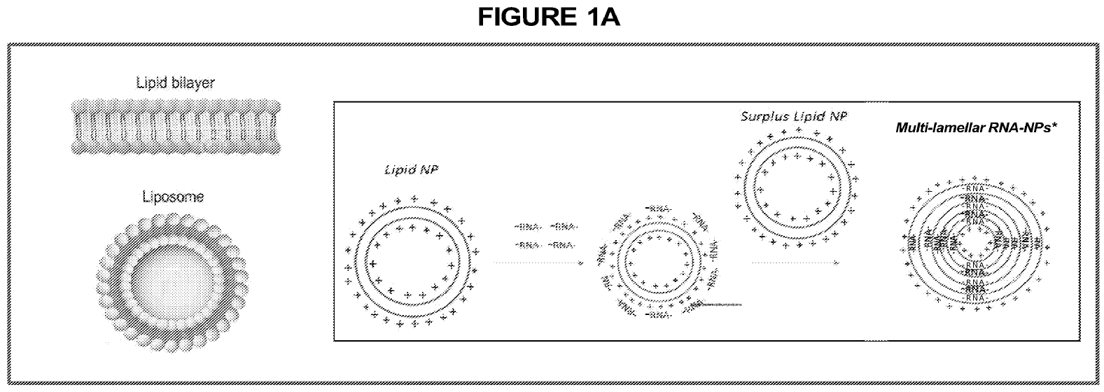

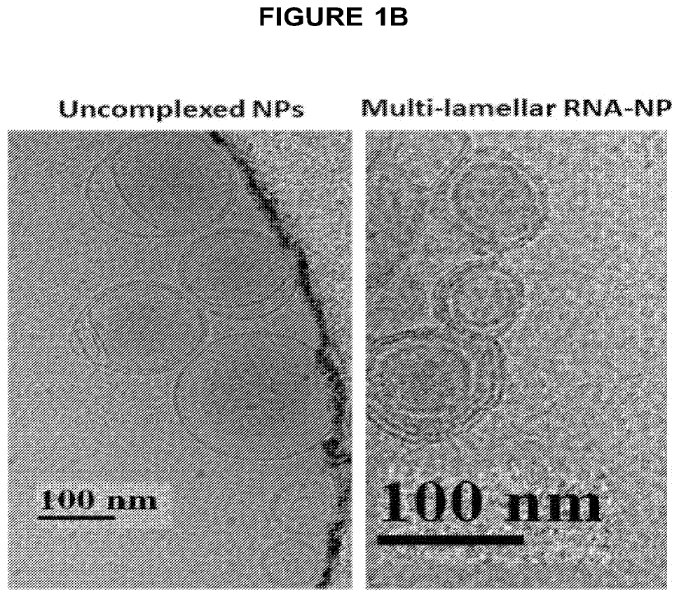

The DOTAP lipid NPs were complexed with RNA to make multilamellar RNA-NPs which were designed to have several layers of mRNA contained inside a tightly coiled liposome with a positively charged surface and an empty core (FIG. 1A). Briefly, in a safety cabinet, RNA was thawed from −80° C. and then placed on ice, and samples comprising PBS and DOTAP (e.g., DOTAP lipid NPs) were brought up to room temperature. Once components were prepared, the desired amount of RNA was mixed with PBS in a sterile tube. To the sterile tube containing the mixture of RNA and PBS, the appropriate amount of DOTAP lipid NPs was added without any physical mixing (without e.g., inversion of the tube, without vortexing, without agitation). The mixture of RNA, PBS, and DOTAP was incubated for about 15 minutes to allow multilamellar RNA-NP formation. After 15 min, the mixture was gently mixed by repeatedly inverting the tube. The mixture was then considered ready for systemic (i.e., intravenous) administration.

The amount of RNA and DOTAP lipid NPs (liposomes) used in the above preparation is pre-determined or pre-selected. In some instances, a ratio of about 15 μg liposomes per about 1 μg RNA were used. For instance, about 75 μg liposomes are used per ˜5 μg RNA or about 375 μg liposomes are used per ˜25 μg RNA. In other instances, about 7.5 μg liposomes were used per 1 μg RNA. Thus, in exemplary instances, about 1 μg to about 20 μg liposomes are used for every μg RNA used.

### Example 2

This example describes the characterization of the nanoparticles of the present disclosure.

### Cryo-Electron Microscopy (CEM)

CEM was used to analyze the structure of multilamellar RNA-NPs prepared as described in Example 1 and control NPs devoid of RNA (uncomplexed NPs) which were made by following all the steps of Example 1, except for the steps under “RNA Preparation” and “Preparation of Multilamellar RNA nanoparticles (NPs)”. CEM was carried out as essentially described in Sayour et al., Nano Lett 17(3) 1326-1335 (2016). Briefly, samples comprising multilamellar RNA-NPs or control NPs were kept on ice prior to being loaded in a snap-freezed in Vitrobot (and automated plunge-freezer for cryoTEM, that freezes samples without ice crystal formation, by controlling temperature, relative humidity, blotting conditions and freezing velocity). Samples were then imaged in a Tecnai G2 F20 TWIN 200 kV/FEG transmission electron microscope with a Gatan UltraScan 4000 (4 k×4 k) CCD camera. The resulting CEM images are shown in FIG. 1B. The right panel is a CEM image of multilamellar RNA-NPs and the left panel is a CEM image of control NPs (uncomplexed NPs). As shown in FIG. 1B, the control NPs contained at most 2 layers, whereas multilamellar RNA NPs contained several layers.

### Zeta Potentials

Zeta potentials of multilamellar RNA NPs were measured by phase analysis light scattering (PALS) using a Brookhaven ZetaPlus instrument (Brookhaven Instruments Corporation, Holtsville, NY), as essentially described in Sayour et al., Nano Lett 17(3) 1326-1335 (2016). Briefly, uncomplexed NPs or RNA-NPs (200 μL) were resuspended in PBS (1.2 mL) and loaded in the instrument. The samples were run at 5 runs per sample, 25 cycles each run, and using the Smoluchowski model.

The zeta potential of the multilamellar RNA NPs prepared as described in Example 1 was measured at about +50 mV. Interestingly, this zeta potential of the multilamellar RNA NPs was much higher than those described in Sayour et al., Oncoimmunology 6(1):e1256527 (2016), which measured at around +27 mV. Without being bound to any particular theory, the way in which the DOTAP lipid NPs are made for use in making the multilamellar RNA NPs (Example 1) involving a vacuum-seal method for evaporating off chloroform leads to less environmental oxidation of the DOTAP lipid NPs, which, in turn, may allow for a greater amount of RNA to complex with the DOTAP NPs and/or greater incorporation of RNA into the DOTAP lipid NPs.

### RNA Incorporation by Gel Electrophoresis:

A gel electrophoresis experiment was conducted to measure the amount of RNA incorporated into ML liposomes. Based on this experiment, it was qualitatively shown that nearly all, if not all, of the RNA used in the procedure described in Example 1 was incorporated into the DOTAP lipid NPs. Additional experiments to characterize the extent of RNA incorporation are carried out by measuring RNA-NP density and comparing this parameter to that of lipoplexes.

### Example 3

This example describes a comparison of the nanoparticles of the present disclosure to cationic RNA lipoplexes and anionic RNA lipoplexes.

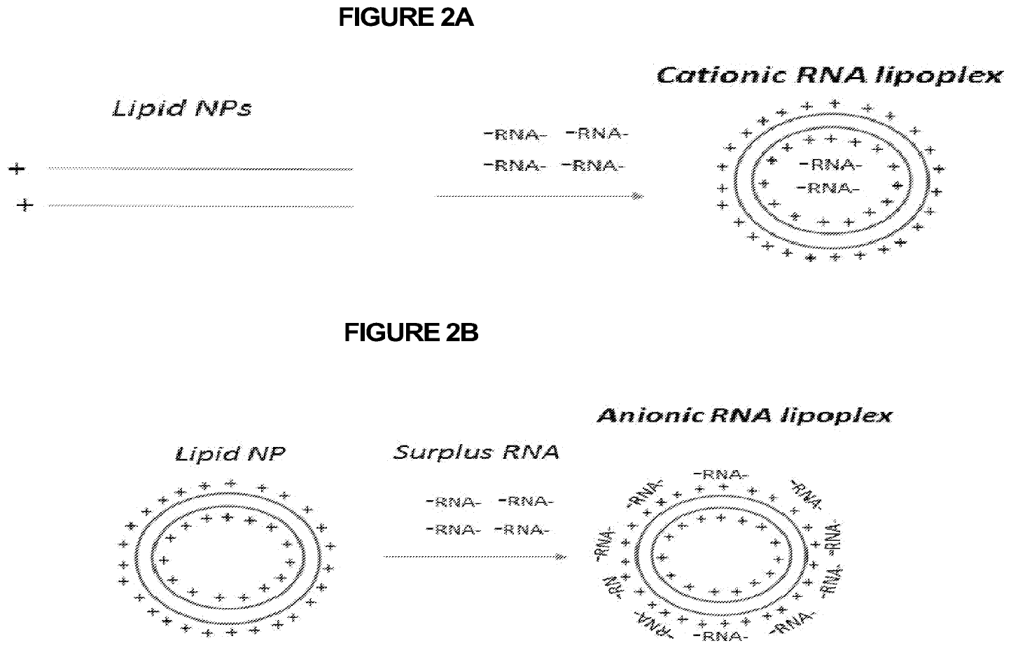

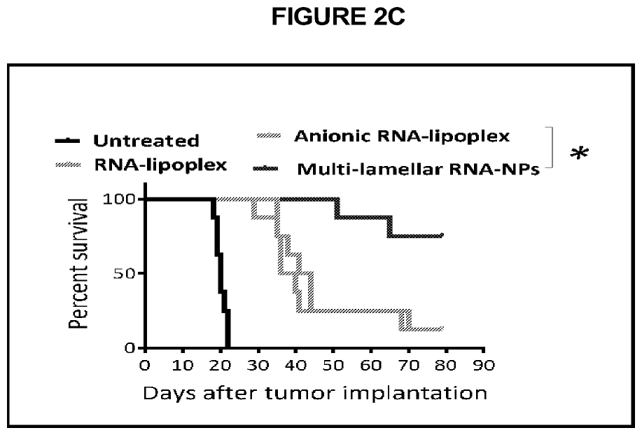

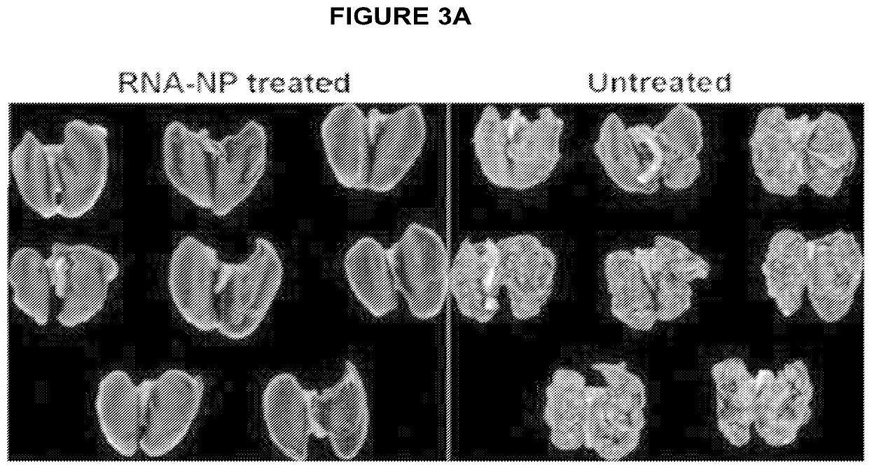

Cationic lipoplexes (LPX) were first developed with mRNA in the lipid core shielded by a net positive charge located on the outer surface (FIG. 2A). Anionic RNA lipoplexes (FIG. 2B) have been developed with an excess of RNA tethered to the surface of bi-lamellar liposomes. RNA-LPX were made by mixing RNA and lipid NP at ratios to equalize charge. Anionic RNA-NPs were made by mixing RNA and lipid NP at ratios to oversaturate lipid NPs with negative charge. Various aspects of the RNA-LPX and anionic RNA LPX were then compared to the multilamellar RNA NPs described in the above examples.

Cryo-Electron Microscopy (CEM) was used to compare the structures of the RNA LPX and the multilamellar RNA-NPs prepared as described in Example 1. Uncomplexed NPs were used as a control. CEM was carried out as essentially described in Example 2. The data support that more RNA is held by the ML RNA-NPs. Additional data show that the concentration drops more with ML RNA-NP complexation versus RNA LPX supporting multilamellar formation of ML RNA-NPs not observed by simple mixing of equivalent amounts of RNA and lipid NPs by mass or charge (i.e., RNA-LPX and anionic RNA-LPX respectively). This supports that more RNA is “held” by ML RNA-NPs.

Also, an experiment was conducted to determine where the anionic LPXs localize upon administration to mice. Anionic LPXs localized to the spleens of animals upon administration.

RNA LPX, anionic lipoplex (LPX), or multilamellar RNA-NPs were administered to mice and spleens were harvested one week later for assessment of activated DCs (*p<0.05 unpaired t test). The RNA used in this experiment was tumor-derived mRNA from the K7M2 tumor osteosarcoma cell line. Mice treated with multilamellar RNA NPs exhibited the highest levels of activated DCs.

Anionic tumor mRNA-lipoplexes, tumor mRNA-lipoplexes, and multilamellar tumor mRNA loaded NPs were compared in a therapeutic lung cancer model (K7M2) (n=5-8/group). Each vaccine was intravenously administered weekly (×3) (**p<0.01, Mann Whitney). The data demonstrated that ML RNA-NPs allow for substantially greater innate immunity which is enough to drive efficacy from even non-antigen specific ML RNA-NPs. The data also demonstrated the superior efficacy of multilamellar tumor specific RNA-NPs, relative to anionic LPX and RNA LPX.

Anionic tumor mRNA-lipoplexes, cationic tumor mRNA-lipoplexes and multilamellar tumor mRNA loaded NPs were compared in a therapeutic lung cancer model (K7M2) (n=8/group). Each vaccine was i.v. administered weekly (×3), *p<0.05, Gehan Breslow-Wilcoxon test. The percent survival was measured by Kaplan-Meier Curve analysis. As shown in FIG. 2C, multilamellar tumor specific RNA-NPs mediated superior efficacy, compared to cationic RNA lipoplexes and anionic RNA lipoplexes, for increasing survival.

Herein it is demonstrated that the multilamellar RNA-NP formulation targeting physiologically relevant tumor antigens is more immunogenic and significantly more efficacious compared with anionic LPX and RNA LPX. Without being bound to any particular theory, by altering RNA-lipid ratios and increasing the zeta potential, a novel RNA-NP design composed of multi-lamellar rings of tightly coiled mRNA has been developed, which multi-lamellar design is thought to facilitate increased NP uptake of mRNA (condensed by alternating positive/negative charge) for enhanced particle immunogenicity and widespread in vivo localization to the periphery and tumor microenvironment (TME). Systemic administration of these multi-lamellar RNA-NPs localize to lymph nodes, reticuloendothelial organs (i.e., spleen and liver) and to the TME, activating DCs therein (based on increased expression of the activation marker CD86 on CD11c+ cells). These activated DCs prime antigen specific T cell responses, which lead to anti-tumor efficacy (with increased TILs) in several tumor models.

### Example 4

This example demonstrates the ability of multilamellar RNA-NPs to systemically activate DCs, induce antigen specific immunity, and elicit anti-tumor efficacy.

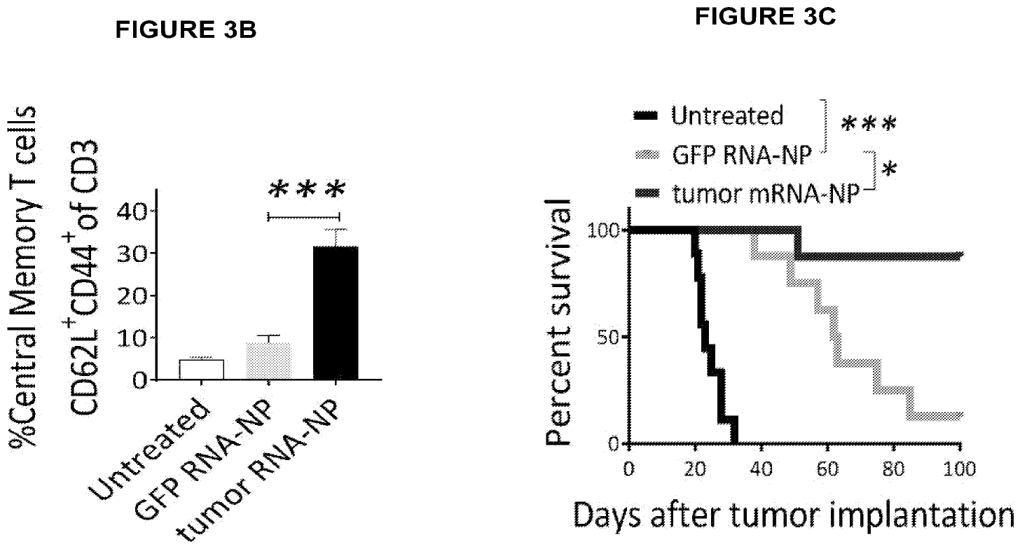

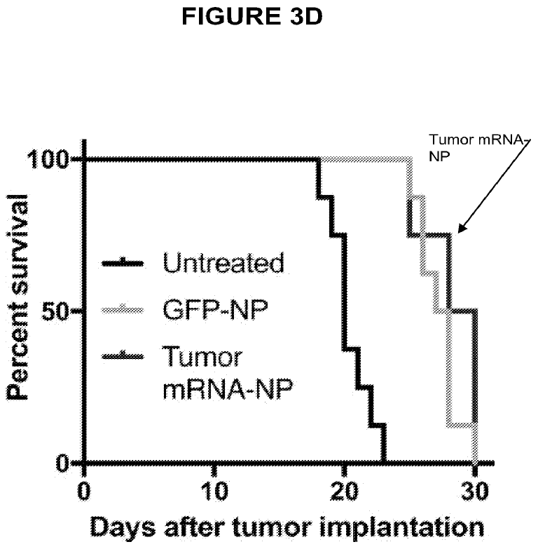

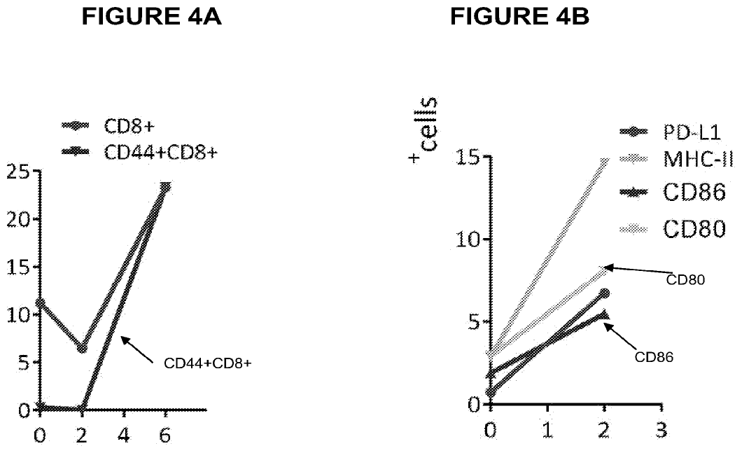

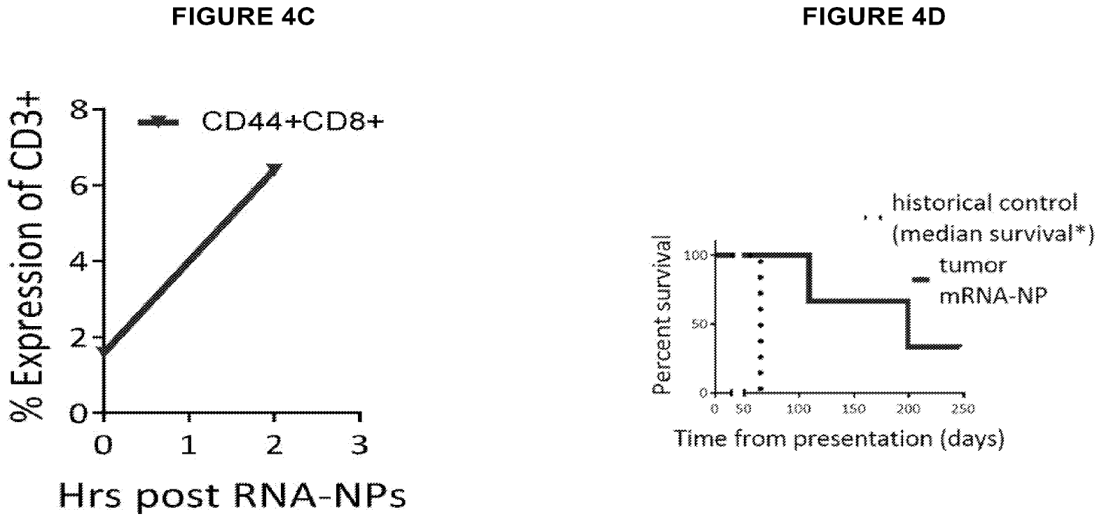

The effect of multilamellar RNA NPs were tested in a second model. Here, BALB/c mice (8 mice per group) inoculated with K7M2 lung tumors were vaccinated thrice-weekly with multilamellar RNA-NPs. A control group of mice was untreated. The lungs were harvested one week after the 3rd vaccine for analysis of intratumoral memory T cells ***p<0.001, Mann Whitney test. FIG. 3A provides a pair of photographs of RNA-NP treated-lungs (left) and of untreated lungs (right). FIG. 3B is a graph of the % central memory T cells (CD62L+CD44+ of CD3+ cells) in the harvested lungs of untreated mice, mice treated multilamellar RNA NPs with GFP RNA, and mice treated multilamellar RNA NPs with tumor-specific RNA.

Also, BALB/c mice or BALB/c SCID (Fox Chase) mice (8 mice per group) were inoculated with K7M2 lung tumors and vaccinated intravenously thrice-weekly with multilamellar RNA-NPs comprising GFP RNA or tumor-specific RNA. A control group of mice was untreated. % survival was plotted on a Kaplan-Meier curve (***p<0.0001, Gehen-Breslow-Wilcox). As shown in FIG. 3C, the percent survival of BALB/c mice treated with multilamellar RNA NPs with tumor-specific RNA was highest among the three groups. Interestingly, the percent survival of BALB/c SCID (Fox Chase) mice treated with multilamellar RNA NPs with GFP RNA was about the same as mice treated with multilamellar RNA NPs with tumor-specific RNA (FIG. 3D).

Taken together, the data of FIGS. 3A-3D demonstrate that monotherapy with RNA-NPs comprising GFP RNA or tumor-specific RNA mediates significant anti-tumor efficacy against metastatic lung tumors in immunocompetent animals and SCID mice. In BALB/c mice bearing metastatic lung tumors (FIG. 3A-3D), both GFP (control) and tumor specific RNA-NPs mediate innate immunity and anti-tumor activity; however, only tumor specific RNA-NPs mediate increases in intratumoral memory T cells and long-term survivor outcome (FIG. 3A-3D). Anti-tumor activity of RNA-NPs in mice bearing intracranial malignancies was also demonstrated (data not shown).

These data demonstrate that multilamellar RNA-NPs systemically activate DCs, induce antigen specific immunity and elicit anti-tumor efficacy. FIGS. 3A-3D shows that control RNA-NPs elicit innate response with some efficacy that is not as robust as tumor specific RNA-NPs. Compared with untreated mice, no effects of uncomplexed NPs have been observed, but both non-specific (GFP RNA) and tumor-specific RNA when incorporated into multilamellar RNA NPs mediate innate immunity; however only tumor specific RNA-NPs elicit adaptive immunity that results in a long-term survival benefit (FIG. 3A-3D).

### Example 5

This example demonstrates personalized tumor RNA-NPs are active in a translational canine model.

The safety and activity of multilamellar RNA-NPs was evaluated in client-owned canines (pet dogs) diagnosed with malignant gliomas or osteosarcomas. The malignant gliomas or osteosarcomas from dogs were first biopsied for generation of personalized tumor RNA-NP vaccines.

To generate personalized multilamellar RNA NPs, total RNA materials was extracted from each patient's biopsy. A cDNA library was then prepared from the extracted total RNA, and then mRNA was amplified from the cDNA library. mRNA was then complexed with DOTAP lipid NPs, into multilamellar RNA-NPs as essentially described in Example 1. Blood was drawn at baseline, then 2 hours and 6 hours post-vaccination for assessment of PD-L1, MHCII, CD80, and CD86 on CD11c+ cells. CD11c expression of PD-L1, MHC-II, PDL1/CD80, and PD-L1/CD86 is plotted over time during the canine's initial observation period. CD3+ cells were analyzed over time during the canine's initial observation period for percent CD4 and CD8, and these subsets were assessed for expression of activation markers (i.e., CD44). From these data, it was shown that multilamellar RNA-NPs elicited an increase in 1) CD80 and MHCII on CD11c⁺ peripheral blood cells demonstrating activation of peripheral DCs; and 2) an increase in activated T cells.

Interestingly, within a few hours after administration, tumor specific RNA-NPs elicited margination of peripheral blood mononuclear cells, which increased in the subsequent days and weeks post-treatment; suggesting that RNA-NPs mediate lymphoid honing of immune cell populations before egress.

These data demonstrated that personalized mRNA-NPs are safe and active in translational canine disease models.

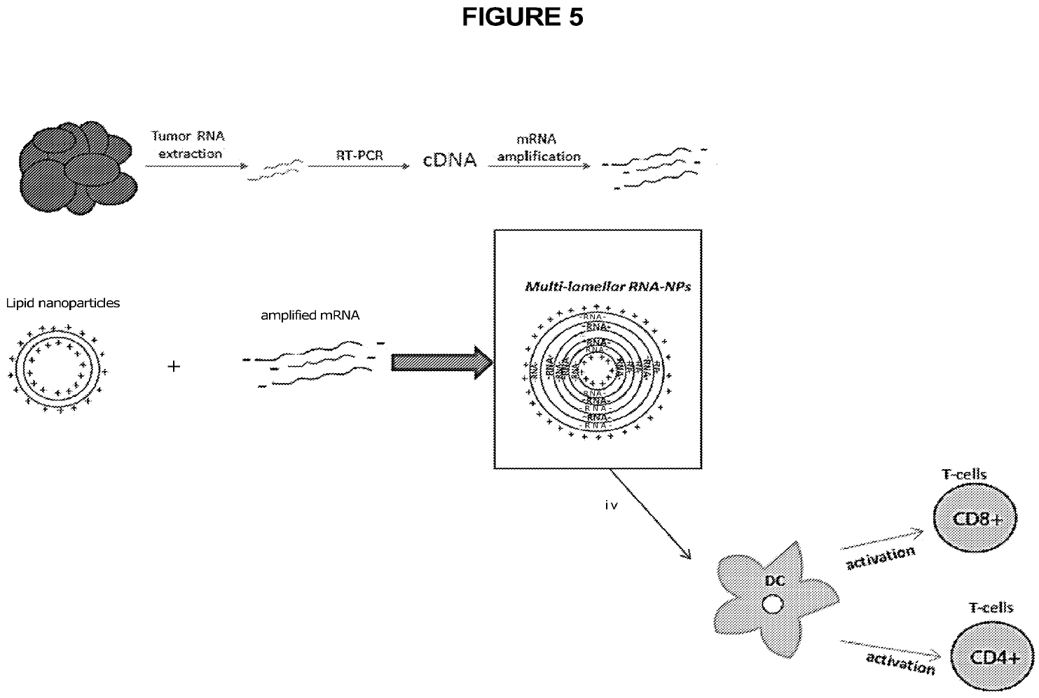

Specific data from canines evaluated in this manner are shown. A 31 kg male Irish Setter was enrolled on study per owner's consent to receive multilamellar RNA-NPs. Tumor mRNA was successfully extracted and amplified after tumor biopsy. Immunologic response was plotted in response to 1^{st} vaccine. The data show increased activation markers over time on CD11c+ cells (DCs) (FIG. 4A), The data show increased CD8+ cells that are activated (CD44+CD8+ cells) within the first few hours post RNA-NP vaccine. These data support that the multilamellar RNA-NPs are immunologically active in a male Irish Setter. A male boxer diagnosed with a malignant glioma was enrolled on study per owner's consent to receive RNA-NPs. Tumor mRNA was successfully extracted and amplified after tumor biopsy. Immunologic response is plotted in response to 1^{st} vaccine (FIG. 4B). The data show increased activation markers over time on CD11c+ cells (DCs). As shown in FIG. 4C, an increase in activated T cells (CD44+CD8+ cells) was observed within the first few hours post RNA-NP vaccine. These data support that the multilamellar RNA-NPs are immunologically active in a male canine boxer.

After receiving weekly RNA-NPs (×3), the canines diagnosed with malignant gliomas had a steady course. Post vaccination MRI showed stable tumor burdens, with increased swelling and enhancement (in some cases), which may be more consistent with pseudoprogression from an immunotherapeutic response in otherwise asymptomatic canines. Survival of canines diagnosed with malignant gliomas receiving only supportive care and tumor specific RNA-NPs (following tumor biopsy without resection) is shown in FIG. 4D. In FIG. 4D, the median survival (shown as dotted line) was about 65 days and was reported from a meta-analysis of canine brain tumor patients receiving only symptomatic management. In a previous study, cerebral astrocytomas in canines has been reported to have a median overall survival of 77 days. The personalized, multilamellar RNA NPs allowed for survival past 200 days.

Aside from low-grade fevers that spiked 6 hrs post-vaccination on the initial day, personalized tumor RNA-NPs (1×) were well tolerated with stable blood counts, differentials, renal and liver function tests. To date, we have treated four canines diagnosed with malignant brain tumors. It is important to highlight that these canines received no other therapeutic interventions for their malignancies (i.e., surgery, radiation or chemotherapy), and all patients assessed developed immunologic response with pseudoprogression or stable/smaller tumors. Significant toxicities in canines have not been observed that would preclude investigation in humans at 1× dosing based on clinical presentation, physical exam findings, and laboratory tests. One canine was autopsied after RNA-NP vaccines. In this patient, there were no toxicities believed to be related to the interventional agent.

These results suggest safety and activity of tumor specific RNA-NPs in client-owned canines with malignant brain tumors for subjects that did not receive any other anti-tumor therapeutic interventions.

### Example 6

This example demonstrates non-antigen specific multilamellar (ML) RNA NPs mediate antigen-specific immunity long enough to confer memory and fend off re-challenge of tumor.

An experiment was carried out with long-term surviving mice (e.g., mice that survived for ˜100 days) that were challenged a total of two times via tumor inoculation, but treated only once weekly (×3) with ML RNA NPs comprising GFP RNA or pp65 RNA (each of which were non-specific to the tumor) or with ML RNA NPs comprising tumor-specific RNA. The treatment occurred just after the first tumor inoculation and about 100 days before the second tumor inoculation. Because none of the control mice (untreated mice) survived to 100 days, a new control group of mice were created by inoculating the same type of mice with K7M2 tumors. The new control group like the original control mice did not receive any treatment. The long-time survivors also did not receive any treatment after the second time of tumor inoculation.

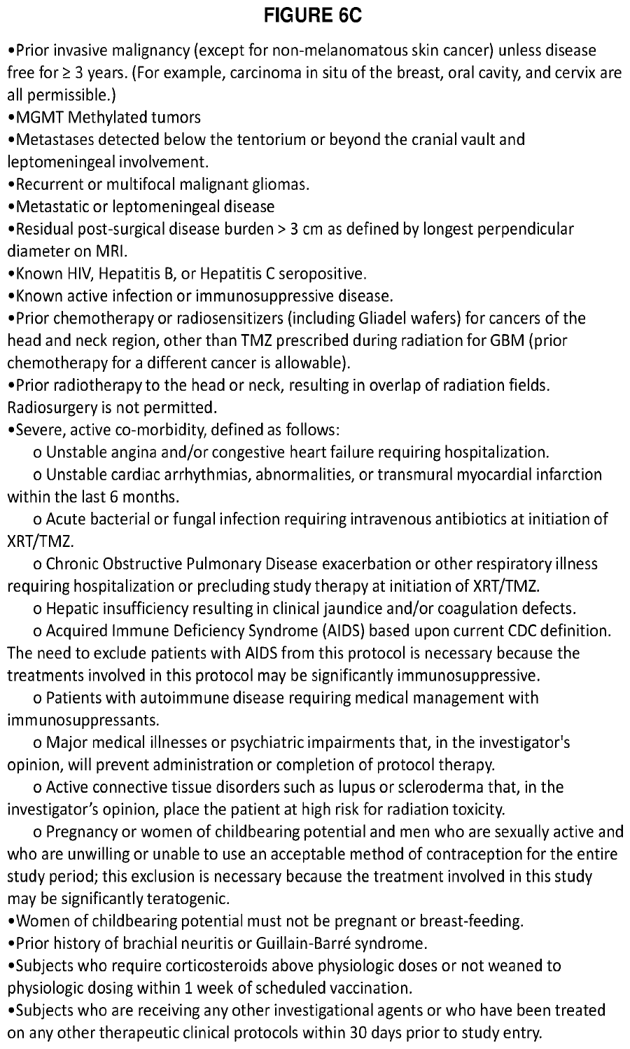

Remarkably, mice in all three groups contained long-time survivors that survived the second tumor challenge. As shown in FIG. 7 (which shows only the time period following the 2^{nd} inoculation), mice in all three groups contained long-time survivors with survival to 40 days post tumor implantation (second instance of tumor inoculation). Interestingly, the percentage of long-time survivor mice that were previously treated with ML RNA NPs comprising non-specific RNA (GFP RNA or pp65 RNA) survived to 40 days post second tumor inoculation, comparable to the group treated with ML RNA NPs comprising tumor specific RNA (treated before second tumor challenge).

These data support that ML RNA NPs comprising RNA non-specific to a tumor in a subject provides therapeutic treatment for the tumor comparable to that provided by ML RNA NPs comprising RNA specific to the tumor, leading to increased percentage in animal survival.

### Example 7

This example describes a method of administering the nanoparticles of the present disclosure to a human subject. In particular, this example describes a study characterizing the safety and immunologic activity of tumor antigen-LP vaccines in adults.

This study represents a first in human Phase I/II study of RNA-LP vaccines for newly diagnosed adult MGMT unmethylated glioblastoma (GBM) and pediatric high-grade gliomas (pHGG). The first phase of the study involves a dose-escalation study using the BOIN design with an initial embedded accelerated titration design (ATD) to efficiently identify the maximally tolerated dose (MTD). One object is to confirm the manufacturing feasibility and safety, and to confirm the MTD of RNA-LP vaccines in adult patients with newly diagnosed MGMT unmethylated GBM and patients with newly diagnosed pHGG.

The trial will consist of three parts: Surgery, Radiation, and Immunotherapy.

Surgery. Potentially eligible subjects will be enrolled on a screening consent for the sterile collection of tumor material in a manner suitable for RNA extraction, amplification, and loading of lipid particles (LPs). Following surgical resection with confirmatory pathologic diagnosis (at local institution), patients will be enrolled in the trial after informed consent has been obtained. Meanwhile investigators will generate tumor specific RNA-LP vaccines that will be shipped back to the enrolling site.

Radiation: Radiotherapy should begin within 4 weeks (+/−14 days) of surgery or sooner based on institutional preference. Standard external beam RT is administered concomitantly with TMZ (adult GBM patients). Institutional practices for administration of external beam RT for subjects with GBM may be followed. Forty-two doses of temozolomide (TMZ) 75 mg/m²/day will be given continuously during radiation for up to 49 days to account for delays in radiation treatment in adult GBM subjects. Delays or discontinuations of TMZ may be necessary due to ongoing clinical and laboratory assessment and tolerability of concomitant TMZ. Since eligibility is restricted to MGMT unmethylated primary adult type GBM, patients will not receive adjuvant cycles of temozolomide.

Radiation guidelines for adult patients: Standard external beam RT is administered. Institutional practices for administration of external beam RT for subjects with adult MGMT unmethylated GBM may be followed. Otherwise the following guidelines should be used:

Target volume definition: (a) Gross Tumor Volume (GTV): The GTV includes all gross residual tumor and/or the tumor bed as defined by MR imaging and operative report. The GTV in many cases will involve a contracted or collapsed tumor bed. Tissue defects resulting from surgical approaches will not be included as part of the GTV when not previously involved by tumor. (b) Clinical Target Volume (CTV): The CTV is meant to treat subclinical microscopic disease and will be an anatomically constrained 5-10 mm margin on the GTV, with additional expansion as necessary to encompass areas of T2/FLAIR change suspicious for tumor involvement prior to surgery. The CTV is limited to the confines of the bony calvarium, falx and tentorium where applicable and extends up to but not beyond neuroanatomic structures through which tumor extension or invasion is certain not to have occurred. When the GTV approaches the boundary of an anatomic compartment, the CTV will extend up to and include the boundary.

Planning target volume 1 (PTV1): CTV+3 mm; Planning target volume 2 (PTV2): GTV+3 mm.

Total dose: PTV1-46 Gy at 2Gy/fraction or 45-50.4 Gy at 1.8 Gy/fraction; PTV2-59.4-60 Gy.

Dose per fraction: 1.8-2.0 Gy/fx daily, five days per week. PTV1 and PTV2 should be delivered in sequential phases over 30-33 fractions.

Radiation guidelines for pediatric patients: Standard external beam RT is administered. Institutional practices for administration of external beam RT for subjects with pHGG may be followed. Otherwise the following guidelines should be used:

One treatment of 1.8-2.0 Gy/fraction is given daily 5 days per week for a total of 59.4-60.0 Gy over <7 weeks. 3D conformal and intensity-modulated RT is permitted. All portals should be treated during each treatment session. Doses are specified as the target dose that shall be to the center of the target volume.

The gross target volume (GTV) for both the initial volume (GTV1) and the conedown volume (GTV2) should be based on the postoperative CT/MRI (and preferably the MRI; the preoperative scans may be used if postoperative scans are not available). This initial target volume (GTV1) should include the contrast-enhancing lesion (and should include the surgical resection cavity) and surrounding edema (if it exists) demonstrated on CT/MRI plus a 2.0-cm margin (this 2.0-cm margin-extended volume will be considered the initial planning target volume, or PTV1). The initial target volume will be treated to 46 Gy at 2Gy/fraction or 45-50.4 Gy at 1.8Gy/fraction. If no surrounding edema is present, the initial planning target volume (PTV1) should include the contrast-enhancing lesion (and should include the surgical resection cavity) plus a 2.5-cm margin. Clinical judgment may be used to modify PTV1 to exclude sensitive structures such as the optic chiasm, non-cranial contents, or anatomic regions in the brain where natural barriers would likely preclude microscopic tumor extension, such as the cerebellum, the contralateral hemisphere, directly across from the tentorium cerebri, the ventricles, etc. After 46 Gy, the tumor volume (GTV2) for the conedown treatment should include the contrast-enhancing lesion (without edema) on the pre-surgery CT/MRI scan plus a 1.5-2-cm margin (PTV2). Treat to 14 Gy at 2Gy/fraction or 14.4-9.0 Gy at 1.8Gy/fraction to a total of 60.0 or 59.4Gy, respectively.

Dose is prescribed to the isodose line such that at least 95% of the target volume receives the prescribed dose.

The optic apparatus should be limited to a maximum of 54Gy and no more than 5% of the volume of the brainstem should receive >54Gy.

Radiation is delayed or interrupted if the platelet count is <20,000. Radiation will not begin or resume until the platelet count is ≥20,000. Hematologic toxicities should be rated on a scale of 0-5 as defined in the CTCAE 5.0.

Immunotherapy: RNA-LP administration begins within four weeks following radiation pending recovery of peripheral blood counts (i.e., ANC >1500/μL, platelets >150/μL), and after assessment of post-radiation MRI (for baseline). Higher thresholds are set since repeated RNA-NP administrations may elicit peripheral blood cytopenias. After radiation, patients will receive three RNA-LP vaccines every two weeks before beginning 12 cycles of adjuvant monthly RNA-LP vaccines for a total of 15 administrations.

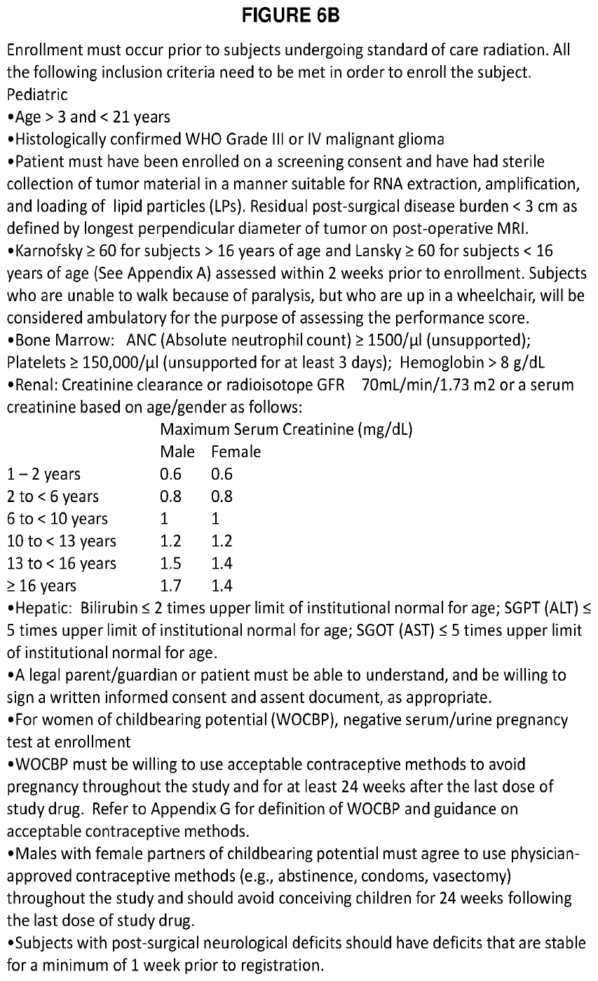

Subjects: A maximum of 28 adult patients will be enrolled in the dose-escalation study using the Bayesian optimal interval (BOIN) design with an initial embedded accelerated titration design (ATD). A maximum of 24 pediatric patients will be enrolled in a dose-escalation study using the BOIN design. Inclusion and exclusion criteria are provided in FIGS. 6A-6D.

### Agent Administration

TMZ: Subjects are administered (orally) temozolomide (TMZ), 75 mg/m²/day, during radiation. Administration occurs on day 1 radiation therapy and proceeds for a maximum of 42 days. Generally, TMZ is taken on an empty stomach (at least one hour before or two hours after food) at approximately the same time each day of radiation and weekends during radiation. Preferably, administer at bedtime on an empty stomach (at least 1 hour before or 2 hours after food) to decrease nausea and vomiting and improve absorption. The whole dose, even if comprised of several capsule sizes, should be taken at one time at approximately the same time each day. Temozolomide dosing should be performed following institutional practice (e.g. +/−5 to 10% of the calculated dose). It is recommended that antiemetics be given 30 minutes prior to each temozolomide dose. If emesis occurs within 20 minutes of taking a given dose, then the dose may be repeated once. If emesis occurs after 20 minutes, the dose should not be repeated. Oral suspension may be compounded if unable to swallow capsules. While receiving TMZ, subjects should receive PCP prophylaxis per institutional guidelines.

If radiotherapy has to be temporarily interrupted for technical or medical reasons unrelated to the temozolomide administration, then treatment with daily temozolomide should continue. If radiotherapy has to be permanently interrupted, then treatment with daily temozolomide should stop.

The 42 days of temozolomide should be given regardless of the end date of RT. If a dose of temozolomide is given and radiation therapy is NOT administered due to sedation or technical issues, the temozolomide doses should not be made up (i.e., no more than 42 doses of temozolomide should be given).

RNA-LP: The components for RNA-LP vaccines are personalized tumor mRNA, pp65 fl LAMP mRNA, and DOTAP liposomes. Pp65 fl LAMP mRNA is a messenger ribonucleic acid encoding for the CMV matrix protein pp65 which is expressed in select HGGs and can be used to track adaptive immunity generated by RNA-liposomes. While not wishing to be bound by any particular theory, fl LAMP assists with shuttling epitopes preferentially through the class II presentation pathway. These are referred to herein as RNA-LP. RNA-LP are administered intravenously (IV). A dose of RNA-LPs is administered every two weeks for an initial period of treatment lasting six weeks (three doses over six weeks, optionally on days 1, 15, and 29 of treatment), which is followed by 12 cycles of adjuvant monthly RNA-LP doses for a total of 15 administrations. A dose of RNA-LPs is administered IV at a rate of 0.04 mg/kg/hr, with appropriate flush volume.

Subjects receive one of the doses of RNA-LP in Table 1 per administration.

TABLE 1

| Dose | Dose |
|---|---|
| Dose −4 | 0.000625 mg/kg of mRNA encapsulated in 0.009375 mg/kg LPs |
| Dose −3 | 0.00125 mg/kg mRNA encapsulated in 0.01875 mg/kg LPs |
| Dose −2 | 0.0025 mg/kg mRNA encapsulated in 0.0375 mg/kg LPs |
| Dose −1 | 0.005 mg/kg mRNA encapsulated in 0.075 mg/kg LPs |
| Dose 0 | 0.01 mg/kg mRNA encapsulated in 0.15 mg/kg LPs |
| Dose +1 | 0.02 mg/kg mRNA encapsulated in 0.3 mg/kg LPs |
| Dose +2 | 0.04 mg/kg mRNA encapsulated in 0.6 mg/kg LPs |
| Dose +3 | 0.08 mg/kg mRNA encapsulated in 1.2 mg/kg LPs |

At dose 0 (or at a lower dose at which a dose limiting toxicity (DLT) was observed), three adult patients are treated with a total of three RNA-LP administrations, administered at an interval of every 2 weeks, after chemo-radiation. After the completion of the DLT period (two weeks after the completion of the first three RNA-LP administrations), if there are no safety concerns, enrollment is opened for a pediatric cohort at the dose 0. If there are safety concerns (i.e., one or more DLTs observed), de-escalation to dose −1 occurs and, barring any safety concerns, that dose is used as the starting dose for Stratum 2 (pediatric subjects). If toxicity assessments allow the pediatric stratum to open, the pediatric and adult phase I studies follow separate dose escalations using the BOIN design and will have separate assessments of MTDs.

While the initial DLT period is evaluated two weeks after completion of the first three RNA-LP administration, before dose escalation (within the individual stratums), DLTs are monitored throughout the course of administrations.

Supportive care may include, but is not limited to, antibiotics, antiemetics, antidiarrheals, topical treatments, blood products, intravenous or oral fluids, electrolyte repletion, and will be used as clinically indicated. For signs and symptoms of pseudoprogression, treatment with bevacizumab per institutional guidelines is allowed to minimize swelling.

### Overall Response Assessment

The overall response assessment takes into account response in both the target and non-target lesion, and the appearance of new lesions, where applicable, according to the criteria described in the table below. The best overall response is the best response recorded from the start of the treatment until disease progression/recurrence (taking as reference for progressive disease the smallest measurements recorded since the treatment started). The subject's best response assignment depends on the achievement of both measurement and confirmation criteria.

Target and Non-target lesion: Tumor dimensions are determined by measurement of the longest tumor dimension and its perpendicular for each target lesion. For most CNS tumors, only one lesion/mass is present and therefore is considered a “target” for measurement/follow up to assess for tumor progression/response. If multiple measurable lesions are present, up to 3 can be selected as “target” lesions. Target lesions should be selected on the basis of size and suitability for accurate repeated measurements. All other lesions are followed as non-target lesions (including CSF positive for tumor cells). The lower size limit of the target lesion(s) should be at least twice the thickness of the slices showing the tumor to decrease the partial volume effect.

Tumor Measurements: Regarding MRI imaging, the sequence that best highlights the tumor (T1 enhanced or T2 weighted or FLAIR images) is chosen to determine response criteria. The same sequence should be used for serial measurements. Response determination is based on a comparison of an area [W (longest diameter of the target lesion)×T (transverse measurement, perpendicular to W)] between the baseline assessment and the study date. For MRI imaging (preferred), the longest diameter can be measured from the axial plane or the plane in which the tumor is best seen or measured. The longest measurement of the tumor is referred to as the width (W). The perpendicular measurements should be determined—transverse (T) measurement, perpendicular to the width in the selected plane. The cystic or necrotic components of a tumor are not considered in tumor measurements. Therefore only the solid component of cystic/necrotic tumors should be measured. If cysts/necrosis composes the majority of the lesion, the lesion may not be “measurable”.

Response Criteria for Target/Non-Target Lesions: Response criteria are assessed in 2 dimensions—the product of W×T. To assess response/progression, the ratio is calculated: W×T (current scan) divided by W×T (reference scan). Development of new disease or progression in any established lesions is considered progressive disease, regardless of response in other lesions—e.g. when multiple lesions show opposite responses, the progressive disease takes precedence.

Complete Response is characterized by the disappearance of all enhancing lesions, determined by two separate observations conducted not less than 4 weeks apart; stable or improved T2/FLAIR non-enhancing lesions; and there can be no appearance of new lesions.

Partial Response is characterized by at least a 50% decrease in the size of enhancing target lesions, taking as reference to the baseline MRI and determined by two separate observations conducted not less than 4 weeks apart; stable or improved T2/FLAIR non-enhancing lesions; and there can be no appearance of new lesions.

Stable Disease is characterized by neither sufficient shrinkage to qualify for complete response or partial response nor sufficient increase to qualify for progressive disease, taking as reference the smallest target size since the treatment started; stable or improved T2/FLAIR non-enhancing lesions; and there can be no appearance of new lesions.

Progressive Disease is characterized by at least a 25% increase in the sum of the size of enhancing target lesions, taking as reference the smallest target size since the treatment started that is confirmed on a 3 month follow-up scan as long as the patient is NOT experiencing significant neurological decline (defined as CTCAE grade 3 or higher). In addition, the appearance of new lesions might be part of an immune response and if the patient is clinically stable, these should be confirmed on a 3-month follow-up scan to assess for true progressive disease versus pseudoprogression.

Duration of overall response is measured from the time measurement criteria are met for CR or PR (whichever is first recorded) until the first date that recurrent or progressive disease is objectively documented (taking as reference for progressive disease the smallest measurements recorded since the treatment started).

The duration of overall CR is measured from the time measurement criteria are first met for CR until the first date that progressive disease is objectively documented.

Duration of stable disease is measured from the start of the treatment until the criteria for progression are met, taking as reference the smallest measurements recorded since the treatment started, including the baseline measurements.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed:

1. A method of treating cancer in a human subject, the method comprising
(a) administering to the human subject a dose of nanoparticles every two weeks for an initial treatment period, then (b) administering a dose of nanoparticles once a month for a subsequent treatment period, the nanoparticles comprising a positively-charged surface and an interior comprising (i) a core and (ii) at least two nucleic acid layers, each nucleic acid layer being positioned between a cationic lipid bilayer,
wherein the nucleic acids are derived from a cancer cell, and
the dose comprises about 0.00050 mg/kg to about 1.5 mg/kg of nucleic acid.

2. The method of claim 1, wherein the nanoparticles comprise at least three nucleic acid layers, each of which is positioned between a cationic lipid bilayer.

3. The method of claim 2, wherein the nanoparticles comprise five or more nucleic acid layers, each of which is positioned between a cationic lipid bilayer.

4. The method of any one of claims 1-3, wherein the nanoparticles comprise a zeta potential of about 40 mV to about 60 mV.

5. The method of claim 4, wherein the nanoparticle comprises a zeta potential of about 50 mV.

6. The method of any one of claims 1-5, comprising nucleic acid molecules and cationic lipid at a ratio of about 1 to about 5 to about 1 to about 20.

7. The method of any one of claims 1-6, wherein the cationic lipid is DOTAP or DOTMA.

8. The method of any one of claims 1-7, wherein the nucleic acid molecules are mRNA molecules.

9. The method of any one of claims 1-8, wherein the nucleic acid of a dose is encapsulated into about 0.008 mg/kg to about 1.5 mg/kg of liposome material.

10. The method of any one of claims 1-9, wherein a single dose comprises about 0.000625 mg/kg to about 0.08 mg/kg nucleic acid.

11. The method of claim 10, wherein a single dose comprises about 0.000625 mg/kg of mRNA, about 0.00125 mg/kg mRNA, about 0.0025 mg/kg mRNA, about 0.005 mg/kg mRNA, about 0.01 mg/kg mRNA, about 0.02 mg/kg mRNA, about 0.04 mg/kg mRNA, or about 0.08 mg/kg mRNA.

12. The method of any one of claims 1-11, wherein the initial treatment period comprises administration of three doses over the course of about four weeks, and the subsequent treatment period comprises administration of doses over the course of about 12 months.

13. The method of any one of claims 1-12, wherein the initial treatment period and the subsequent treatment period total about 18 months.

14. The method of any one of claims 1-13, wherein the nanoparticles are in a composition comprising about 10¹⁰ nanoparticles per mL to about 10¹⁵ nanoparticles per mL.

15. The method of any one of claims 1-14, wherein the human is suffering from a malignant brain tumor.

16. The method of claim 15, wherein the malignant brain tumor is a glioblastoma, medulloblastoma, diffuse intrinsic pontine glioma, or a peripheral tumor with metastatic infiltration into the central nervous system.

17. The method of any one of claims 1-16, wherein the human is an adult subject aged 21 years or older.

18. The method of any one of claims 1-16, wherein the human is a pediatric subject aged less than 21 years.
